(19) United States
(12) Patent Application Publication
(10) Pub. No.: US 2018/0267014 A1
(43) Pub. Date: Sep. 20, 2018

(54) MICROFLUIDIC CHAMBER FOR THE STUDY OF NEUROMUSCULAR JUNCTIONS

(71) Applicant: Ramot at Tel-Aviv University Ltd., Tel-Aviv (IL)

(72) Inventors: Eran PERLSON, Tel-Aviv (IL); Izhak MICHAELEVSKI, Tel-Aviv (IL); Ariel IONESCU, Tel-Aviv (IL); Anton SHEININ, Tel-Aviv (IL)

(21) Appl. No.: 15/925,999

(22) Filed: Mar. 20, 2018

Related U.S. Application Data
(60) Provisional application No. 62/473,540, filed on Mar. 20, 2017.

(51) Int. Cl. G01N 33/487; B01L 3/00; C12M 1/42; C12M 1/34; C12M 3/06; C12M 1/00

(52) U.S. Cl. CPC G01N 33/48728; B01L 3/502715; C12M 1/42; C12M 1/22; C12M 23/16; C12M 23/20; C12M 1/3407

(57) ABSTRACT

A microfluidic chamber, including:
an internal compartment sized and shaped for culturing cells;
an external compartment surrounding at least part of the internal compartment, sized and shaped for culturing cells;
at least one flow path connecting the internal compartment and the external compartment, sized and shaped to allow penetration of cell extensions from the internal compartment into the external compartment, wherein the internal compartment, the external compartment and the at least one flow path are formed on a base layer of the microfluidic chamber;
at least one micro-pattern in the base layer of said microfluidic chamber shaped and sized to align cells cultured in the microfluidic chamber relative to each other.

### RELATED APPLICATIONS

This application claims the benefit of priority under 35 USC § 119(e) of U.S. Provisional Patent Application No. 62/473,540 filed on Mar. 20, 2017, the contents of which are incorporated herein by reference in their entirety.

### FIELD AND BACKGROUND OF THE INVENTION

The present invention, in some embodiments thereof, relates to a microfluidic chamber and, more particularly, but not exclusively, to a microfluidic chamber for the study of synapses.

Elongation of the life expectancy dramatically increased the number of neurodegenerative diseases. The absolute majority of the diseases are incurable, ineffectively responsive to therapeutic treatment, slowly developing over years and devastating. Combination of these mutually aggravating factors also created a significant social-economic burden, particularly in the senescing western society.

Synaptic dysfunction is a very early pathological alteration occurring in the neurodegenerative diseases. The classical examples of neurodegenerative disorders, such as Alzheimer's disease, vascular dementia, Parkinson's disease, Huntington's disease, Creutzfeldt-Jacob disease, depression and alcohol-related dementia are not the only illnesses showing synaptic pathology. Disruption of synapse function occurs as the major pathogenic event linked to clinical manifestations in diseases, triggering mechanisms of which reside beyond the synaptic structures as for Amyotrophic Lateral Sclerosis (ALS), traumatic brain injury etc. Although the molecular mechanisms underlying the various types of neurodegenerative diseases may dramatically differ, involving a wide spectrum of signaling pathways, functional manifestation of these alterations are limited. Namely, disruption of synaptic function can be expressed either by alteration of basal synaptic transmission or/and deterioration of various forms of synaptic plasticity.

Multiplicity of the molecular mechanisms of the disease developments creates a perspective foundation for combinatorial therapeutic approach by development of drugs simultaneously targeting multiple components of the pathogenic molecular network of the disease. Identification of proper combination of drugs requires a high-throughput and highly efficient screening system of drugs effect on synaptic functionality. Yet, while there are huge libraries of small molecules and pharmaceutical agents awaiting to be tested, most if not all functional current approaches of preclinical drug screening studies rely on either cell lines/primary cultures or iPS live/death cell analysis, which can be ineffective in neurodegenerative disease treatment/prevention/moderation, since neuronal death occur only at very advanced stages, while there is nothing to treat anymore.

Therefore, development of high-throughput synapse functionality screen system/device for pharmaceutical or bioactive agents will lead to identification of the beneficial combination of drugs, which will be capable to restore synaptic function and prevent advancement of the neurodegenerative diseases. This functional synapse high-throughput system will be also capable to assess drug effect on the key pathological mechanism of many neurological disorders.

### SUMMARY OF THE INVENTION

Following are some examples of some embodiments of the invention:

### Example 1

A microfluidic chamber, comprising:

an internal compartment sized and shaped for culturing cells;
an external compartment surrounding at least part of said internal compartment, sized and shaped for culturing cells;
a plurality of microgrooves in the base layer of said chamber connecting said internal compartment with said external compartment;
at least one electrode associated with said external compartment, wherein said electrode is sized and positioned to measure electric properties of a cell population cultured in said external compartment.

### Example 2

The microfluidic chamber of example 1, wherein said external compartment is shaped as an arc of at least 180 degrees.

### Example 3

The microfluidic chamber of example 2, wherein said electrode is an arc electrode.

### Example 4

The microfluidic chamber of any one of examples 1 to 3, further comprising at least one electrode associated with said internal compartment for applying an electric field to cells cultured in said internal compartment.

### Example 5

The microfluidic chamber of any one of examples 1 to 3, further comprising an additional set of electrodes associated with said microgrooves for measuring electric properties of cell extensions cultured in said microgrooves, wherein said set of electrodes is electrically isolated from other electrodes of said microfluidic chamber.

### Example 6

The microfluidic chamber of example 5, wherein said set of electrodes is positioned between said electrode of said internal compartment and said electrode of said external compartment.

### Example 7

The microfluidic chamber of any one of the previous examples, wherein said internal compartment is sized and shaped for culturing spinal cord explants or motor neurons.

### Example 8

The microfluidic chamber of example 7, wherein a cross-section of said microgrooves is sized for selective penetration of neuronal extensions emanating from said motor neurons into said microgrooves.

### Example 9

The microfluidic chamber of any one of the previous examples, wherein said external compartment is sized and shaped for culturing muscle cells, myotubes, glia cells, glandular cells or neurons.

### Example 10

The microfluidic chamber of example 7, wherein a length of said microgrooves is in a range of 50-700 μm.

### Example 11

The microfluidic chamber of any one of the previous examples, wherein a bottom surface of said internal compartment and/or said external compartment is coated with at least one organic material to increase cell adhesion to said bottom surface.

### Example 12

The microfluidic chamber of example 11, wherein said organic material is selected from a list of laminin, fibronectin, poly-1-lysine, poly-1-ornithine or matrigel.

### Example 13

The microfluidic chamber of any one of the previous examples, wherein said microfluidic chamber is shaped and sized to be positioned within a cell culturing plate.

### Example 14

The microfluidic chamber of any one of examples 1 to 12, wherein said microfluidic chamber is shaped and sized to be positioned within a well of at least 2-well cell culturing plate.

### Example 15

The microfluidic chamber of any of the previous examples, wherein said microfluidic chamber is round.

### Example 16

A microfluidic chamber, comprising:

at least two spaced-apart compartments sized and shaped for culturing cell populations;

a plurality of microgrooves in the base layer of said chamber connecting a first compartment of said compartments with a second compartment of said compartments, wherein said microgrooves are sized and shaped to allow cell extensions from said first compartment to penetrate into said second compartment;

at least three electrodes configured and positioned to measure electric properties and/or to apply an electric field, wherein a first electrode of said three electrodes is associated with said microgrooves for measuring electric properties of a plurality of said cell extensions and wherein a second electrode is associated with said first compartment and a third electrode is associated with said second compartment.

### Example 17

The microfluidic chamber of example 16, wherein said first compartment is a round compartment.

### Example 18

The microfluidic chamber of example 17, wherein said second compartment is an arc compartment of at least 180 degrees surrounding said first compartment.

### Example 19

The microfluidic chamber of example 17, wherein said second compartment is a ring compartment surrounding said first compartment.

### Example 20

The microfluidic chamber of any one of examples 16 to 19, wherein said first compartment and said second compartment are concentric.

### Example 21

The microfluidic chamber of any one of examples 16 to 20, wherein each electrode of said at least three electrodes is electrically isolated from all the other electrodes.

### Example 22

The microfluidic chamber of any one of examples 16 to 21, wherein a bottom surface of said first and/or second compartments is designed to be coated with at least one organic material to increase cell adhesion to said bottom surface.

### Example 23

The microfluidic chamber of example 22, wherein said organic material is selected from a list of laminin, fibronectin, poly-1-lysine, or poly-1-ornithine.

### Example 24

The microfluidic chamber of any one of examples 16 to 23, wherein said first compartment is shaped and sized for culturing neurons.

### Example 25

The microfluidic chamber of any one of examples 16 to 24, wherein said second compartment is shaped and sized for culturing muscle cells, myotubes, glandular cells, neurons or glia cells.

### Example 26

The microfluidic chamber of example 24, wherein a cross-section of said microgrooves is sized to selectively allow penetration of neuronal extensions or axons emanating from said neurons into said microgrooves.

### Example 27

The microfluidic chamber of any one of examples 16 to 26, wherein said microfluidic chamber is shaped and sized to be positioned within a cell culturing plate.

### Example 28

The microfluidic chamber of any one of examples 16 to 26, wherein said microfluidic chamber is shaped and sized to be positioned within a well of at least 2-well cell culturing plate.

### Example 29

The microfluidic chamber of any one of examples 16 to 28, wherein said at least two-spaced apart compartments are fluidically isolated.

### Example 30

The microfluidic chamber of any one of examples 16 to 29, wherein said at least three electrodes measure said electric properties and/or provide an electric field in synchronization.

### Example 31

A method for measuring electric properties of cultured muscle cells, comprising:

culturing neuronal cells and muscle cells in two spaced-apart compartments; and

measuring electric properties of said muscle cells by a first electrode, and electric properties of neuronal extensions extending from said neurons towards said muscle cells or electric properties of said neuronal cells by a second electrode.

### Example 32

The method of example 31, comprising: applying an electric field to said neuronal cells before said measuring.

### Example 33

The method of example 32, wherein said measuring comprises measuring the electric properties of said muscle cells and/or the neuronal extensions in response to said electric field.

### Example 34

The method of anyone of examples 31 to 33, comprising: seeding said neuronal cells and said muscle cells in said two spaced-apart compartments before said culturing.

### Example 35

The method of example 34, comprising: coating a bottom surface of said two spaced-apart compartments with an organic material to increase cell adhesion to said bottom surface.

### Example 36

The method of example 35, wherein said organic material is selected from a list of laminin, fibronectin, poly-1-lysine, or poly-1-ornithine.

### Example 37

The method of example 31, comprising: providing at least one bioactive agent to muscle cells before said measuring; determining the effect of said at least one bioactive agent on said muscle cells based on said measuring.

### Example 38

A method for screening materials capable of restoring synaptic function, comprising:

providing a first cell population and a second separated cell population, wherein said first cell population is capable of forming synapses with said second separated cell population;

treating said first cell population and/or said second separated cell population with at least one material of said materials;

measuring electric properties of said first cell population and/or of said second separated cell population;

determining functionality of said synapses between said first cell population and said second separated cell population based on the results of said measuring; and

identifying said material for restoring functionality of said synapses based on said determining.

### Example 39

The method of example 38, wherein said treating comprises treating said first cell population and/or said second separated cell population at least twice, each with a different dosage of said material, and wherein identifying comprising identifying said dosage of said material capable of restoring functionality of said synapses.

### Example 40

The method of example 38, wherein said treating comprises treating said first cell population and/or said second separated cell population at least twice, each with a different treatment regime of said material, and wherein identifying comprising identifying said treatment regime of said material capable of restoring functionality of said synapses.

### Example 41

The method of example 38, wherein said first cell population and a second separated cell population are cultured for a desired time period for forming said synapses prior to said measuring.

### Example 42

The method of any one of examples 38 to 40, wherein said material comprises drugs or bioactive agents.

### Example 43

The method of any one of examples 38 to 42, wherein said material is capable of preventing the advancement of neurodegenerative diseases.

Following are some additional examples of some embodiments of the invention:

### Example 1

A microfluidic chamber, comprising:

an internal compartment sized and shaped for culturing cells;

an external compartment surrounding at least part of said internal compartment, sized and shaped for culturing cells;

at least one flow path connecting said internal compartment and said external compartment, sized and shaped to allow penetration of cell extensions from said internal compartment into said external compartment, wherein said internal compartment, said external compartment and said at least one flow path are formed on a base layer of said microfluidic chamber;

at least one micro-pattern in said base layer of said microfluidic chamber shaped and sized to align cells cultured in said microfluidic chamber relative to each other.

### Example 2

The microfluidic chamber example 1, wherein said at least one micro-pattern is positioned in said base layer of said at least one flow path and comprising a plurality of microgrooves shaped and sized to align said cell extensions penetrating through said microgrooves from said internal compartment to said external compartment.

### Example 3

The microfluidic chamber of example 2, wherein said external compartment comprises a micro-patterned surface configured to align cells cultured in said external compartment relative to each other and/or relative to said cell extensions.

### Example 4

The microfluidic chamber of example 3, wherein said micro-patterned surface of said external compartment comprises a plurality of elongated recesses shaped and sized to group muscle cells to form aligned myotubes, wherein at least some of said elongated recesses are parallel relative to each other.

### Example 5

The microfluidic chamber of example 4, wherein a width of said recesses is in a range of 2-10 micron, and wherein a length of said recesses is in a range of 50-2000 micron.

### Example 6

The microfluidic chamber of example 4, wherein said plurality of microgrooves are shaped and sized to allow passage of axons emanating from neuronal cells cultured in said internal compartment towards said myotubes cultured in said external compartment.

### Example 7

The microfluidic chamber of example 3, comprising at least one electrode associated with said external compartment, wherein said electrode comprises said micro-patterned surface and is sized and positioned to measure electric properties of a cell population cultured in said external compartment.

### Example 8

The microfluidic chamber of example 1, wherein said external compartment is shaped as an arc surrounding at least 180 degrees of said internal compartment.

### Example 9

The microfluidic chamber of example 8, wherein said electrode is an arc-shaped electrode.

### Example 10

The microfluidic chamber of example 1, further comprising at least one electrode associated with said internal compartment for applying an electric field to cells cultured in said internal compartment.

### Example 11

The microfluidic chamber of example 2, further comprising an additional set of electrodes associated with said microgrooves for measuring electric properties of said cell extensions cultured in said microgrooves, wherein said set of electrodes is electrically isolated from other electrodes of said microfluidic chamber.

### Example 12

The microfluidic chamber of example 11, wherein said set of electrodes is positioned between said electrode of said internal compartment and said electrode of said external compartment.

### Example 13

The microfluidic chamber of example 2, wherein said internal compartment is sized and shaped for culturing spinal cord explants or motor neurons.

### Example 14

The microfluidic chamber of example 13, wherein a cross-section of said microgrooves is sized for selective penetration of neuronal extensions emanating from said motor neurons into said microgrooves.

### Example 15

The microfluidic chamber of example 1, wherein said external compartment is sized and shaped for culturing muscle cells, myotubes, glia cells, glandular cells or neurons.

### Example 16

The microfluidic chamber of example 2, wherein a length of said microgrooves is in a range of 50-700 μm and a width of said microgrooves is in a range of 1-10 μm.

### Example 17

The microfluidic chamber of example 1, wherein a bottom surface of said internal compartment and/or said external compartment is coated with at least one organic material to increase cell adhesion to said bottom surface and wherein said organic material is selected from a list of laminin, fibronectin, poly-1-lysine, poly-1-ornithine or matrigel.

### Example 18

The microfluidic chamber of example 1, wherein said microfluidic chamber is round and/or shaped and sized to be positioned within a cell culturing plate.

### Example 19

The microfluidic chamber of example 1, wherein said microfluidic chamber is shaped and sized to be positioned within a well of at least 2-well cell culturing plate.

### Example 20

The microfluidic chamber of example 1, comprising living cells and/or cell culturing media

### Example 21

A microfluidic chamber, comprising:

at least two spaced-apart compartments sized and shaped for culturing cell populations;

a plurality of microgrooves in the base layer of said chamber connecting a first compartment of said compartments with a second compartment of said compartments, wherein said microgrooves are sized and shaped to allow cell extensions from said first compartment to penetrate into said second compartment;

at least three electrodes configured and positioned to measure electric properties and/or to apply an electric field, wherein a first electrode of said three electrodes is associated with said microgrooves for measuring electric properties of a plurality of said cell extensions and wherein a second electrode is associated with said first compartment and a third electrode is associated with said second compartment.

### Example 22

A method for measuring electric properties of cultured muscle cells, comprising:

culturing neuronal cells and muscle cells in two spaced-apart compartments; and

measuring electric properties of said muscle cells by a first electrode, and electric properties of neuronal extensions extending from said neurons towards said muscle cells or electric properties of said neuronal cells by at least one second electrode.

### Example 23

The method of example 22, comprising: applying an electric field to said neuronal cells before said measuring.

### Example 24

The method of example 23, wherein said measuring comprises measuring the electric properties of said muscle cells and/or the neuronal extensions in response to said electric field.

### Example 25

The method of example 22, comprising:

providing at least one bioactive agent to muscle cells before said measuring;

determining the effect of said at least one bioactive agent on said muscle cells based on said measuring.

### Example 26

The method of example 22, wherein said culturing comprises culturing said muscle cells within elongated recesses to form aligned and parallel myotubes relative to each other, and wherein measuring comprises measuring electric properties of at least some of said aligned and parallel myotubes.

### Example 27

A method for screening materials capable of restoring synaptic function, comprising:

providing a first cell population and a second separated cell population, wherein said first cell population is capable of forming synapses with said second separated cell population;

treating said first cell population and/or said second separated cell population with at least one material of said materials;

measuring electric properties of said first cell population and/or of said second separated cell population;

determining functionality of said synapses between said first cell population and said second separated cell population based on the results of said measuring; and

identifying said material for restoring functionality of said synapses based on said determining.

### Example 28

The method of example 27, wherein said treating comprises treating said first cell population and/or said second separated cell population at least twice, each with a different dosage of said material, and wherein identifying comprising identifying said dosage of said material capable of restoring functionality of said synapses.

### Example 29

The method of example 27, wherein said treating comprises treating said first cell population and/or said second separated cell population at least twice, each with a different treatment regime of said material, and wherein identifying comprising identifying said treatment regime of said material capable of restoring functionality of said synapses.

### Example 30

The method of example 27, wherein said first cell population and a second separated cell population are cultured for a desired time period for forming said synapses prior to said measuring.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as measuring electric activity of neuromuscular junctions, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings and images makes apparent to those skilled in the art how embodiments of the invention may be practiced.

In the drawings:

FIG. 1A is a flow chart of a process for generation of neuromuscular junctions in culture, according to some embodiments of the invention;

FIG. 1B is a block diagram of a system for synapse generation in culture and for analysis of the synapses, according to some embodiments of the invention;

FIG. 1C is a schematic illustration of a microfluidic chamber for co-culturing neurons and muscle cells, according to some embodiments of the invention;

FIGS. 1D-1F are schematic illustrations of a microfluidic chamber which includes an electrode array, according to some embodiments of the inventions;

FIGS. 1G-1K are schematic illustrations of different cell types that are cultured in the microfluidic chamber, according to some embodiments of the invention;

FIG. 2A is schematic illustration of a compartmental chamber for electrical properties measurements of cultured neurons and muscle cells, according to some embodiments of the invention;

FIG. 2B is a schematic illustration of an assay platform for electrical properties measurements of cultured neurons and muscle cells, according to some embodiments of the invention;

FIG. 2C is a flow chart describing a process of recording and analyzing electrical signals of one or more of muscle cells, myotubes, neuronal cells or axons, according to some embodiments of the invention;

FIG. 2D is a block diagram of an analytical unit, according to some embodiments of the invention;

FIGS. 3A and 3B are schematic illustrations of an analytical unit for electrical properties measurements of cultured neurons and muscle cells, according to some embodiments of the invention;

FIG. 3C is a schematic illustration of a spinal cord explant seeded within an analytical unit, according to some embodiments of the invention;

FIG. 3D is a schematic illustration of neuronal processes crossing through a plurality of microgrooves from a central compartment to a peripheral compartment in the analytical unit shown in FIG. 3C, according to some embodiments of the invention;

FIG. 3E is a schematic illustration of the plurality of microgrooves of the analytical unit shown in FIG. 3C, according to some embodiments of the invention;

FIG. 3F is an image of axons crossing the microgrooves into a chamber of the analytical unit shown in FIG. 3C, according to some embodiments of the invention;

FIGS. 4A and 4B are schematic illustrations of an electrode array associated with a microfluidic chamber, according to some embodiments;

FIGS. 5A-5D are schematic illustrations of a segmented electrode array associated with a microfluidic chamber, according to some embodiments of the invention;

FIG. 6A is a flow chart of a process for electrical properties analysis of neuron muscular junctions (NMJ), according to some embodiments of the invention;

FIG. 6B is a flow chart of a process for electrical properties analysis of synapses, according to some embodiments of the invention;

FIG. 7 is a flow chart of a process for screening drug compounds capable of restoring synaptic function, according to some embodiments of the invention;

FIG. 8A is a block diagram of a micro-patterned electrode, according to some embodiments of the invention;

FIGS. 8B-8D are schematic illustrations of a micro-patterned electrode, according to some embodiments of the invention;

FIGS. 8E and 8F are schematic illustrations of a micro-patterned surface with a plurality of electrodes, according to some embodiments of the invention;

FIG. 9A is a schematic illustration of muscle cells seeded on a non-micropatterned electrode, according to some embodiments of the invention;

FIG. 9B is a schematic illustration of muscle cells seeded on a micro-patterned electrode, according to some embodiments of the invention;

FIG. 9C is a schematic illustration of an electrode array comprising micro-patterned and non-micro-patterned electrodes, according to some embodiments of the invention;

FIG. 9D is a schematic illustration of an analytical unit with micropatterned electrodes, according to some embodiments of the invention;

FIG. 10A is an image showing neuronal processes cultured in an analytical unit, according to some embodiments of the invention; and

FIGS. 10B-10C are electrical recordings from neuronal processes cultured in the analytical unit shown in FIG. 10A, according to some embodiments of the invention.

### DESCRIPTION OF SPECIFIC EMBODIMENTS OF THE INVENTION

The present invention, in some embodiments thereof, relates to a microfluidic chamber and, more particularly, but not exclusively, to a microfluidic chamber for the study of synapses.

A broad aspect of some embodiments of the invention relates to alignment of cultured cells relative to each other and/or with respect to nerve endings, for example axon endings. In some embodiments, the cultured cells are aligned by at least one micro-pattern or alignment structure, for example a channel, groove and/or recess formed optionally in a cell culture chamber, for example a tissue culture plate, a well of a tissue culture plate or any other chamber designed for culturing cells or tissue, optionally in a base layer of the chamber. In some embodiments, the at least one micro-pattern or alignment structure is formed in a base layer of a microfluidic chamber. Optionally, the at least one micro-pattern or alignment structure is formed in the base layer of an internal chamber, in a base layer of an external chamber and/or in a base layer of a flow path connecting the internal and the external chambers. In some embodiments, the cells are aligned relative to the alignment structure or micro-pattern. In some embodiments, the alignment structure or micro-pattern is formed on and/or within a base layer of the cell culture chamber. Alternatively or additionally, the alignment structure or micro-pattern is formed on and/or within an electrode surface, optionally an upper side of an electrode surface positioned inside the cell culture plate.

An aspect of some embodiments of the invention relates to culturing muscle cells in a microfluidic chamber. In some embodiments, the muscle cells are cultured in an external compartment, optionally surrounding at least partially a spaced-apart internal compartment. In some embodiments, the external compartment surrounds at least 25% of the internal compartment, for example 25%, 40%, 50%, 100% or any intermediate, or smaller percentage.

According to some embodiments, an electrode associated with the external compartment measures electric properties, for example voltage, current, changes in voltage or current of the muscle cells. In some embodiments, neurons cultured in the internal compartment innervate the muscle cells, for example to generate neuromuscular junctions (NMJ). Alternatively, the muscle cells are cultured in an internal compartment and the neurons are cultured in an external compartment for example, an external compartment that surrounds the internal compartment.

According to some embodiments, the neurons extend axons or neurites into at least one flow path connecting the internal and the external compartments. In some embodiments, the at least one flow path comprises at least one micro-pattern, for example a plurality of microgrooves at the base layer of the flow path. Optionally, at least one section of the external compartment is isolated from the internal compartments. In some embodiments, the microgrooves surround at least 70% of the internal compartment, optionally a central compartment, for example surround 50%, 60%, 80%, 100% or any intermediate or smaller surrounding percentage of the internal compartment. A possible advantage of surrounding at least 70% of the internal compartment used for culturing neurons is that it allows direction of more axons towards muscle cells or myotubes cultured in an external compartment, which optionally leads to the formation of a larger number of NMJ.

In some embodiments, an electrode associated with the internal compartment, is configured to apply an electric field to the neurons. In some embodiments, the electric field is applied by setting a desired current or by setting a desired range of currents. Additionally or alternatively, the electric field is applied by setting a desired voltage or by setting a desired range of voltages and/or desired waveform and/or duration of stimuli.

In some embodiments, the electrode used for electric field application is extracellular, and is positioned outside of the cells. Optionally the electrode is not in a direct contact with the cells. In some embodiments, the electrode is positioned outside of the cells and is in a direct contact with at least one cell. In some embodiments, application of an electric field between two electrodes optionally causes the electrical current to pass through the cell medium (solution), which may lead to an electrical field change in the cell's surrounding. In some embodiments, the electric field application causes neuronal excitation.

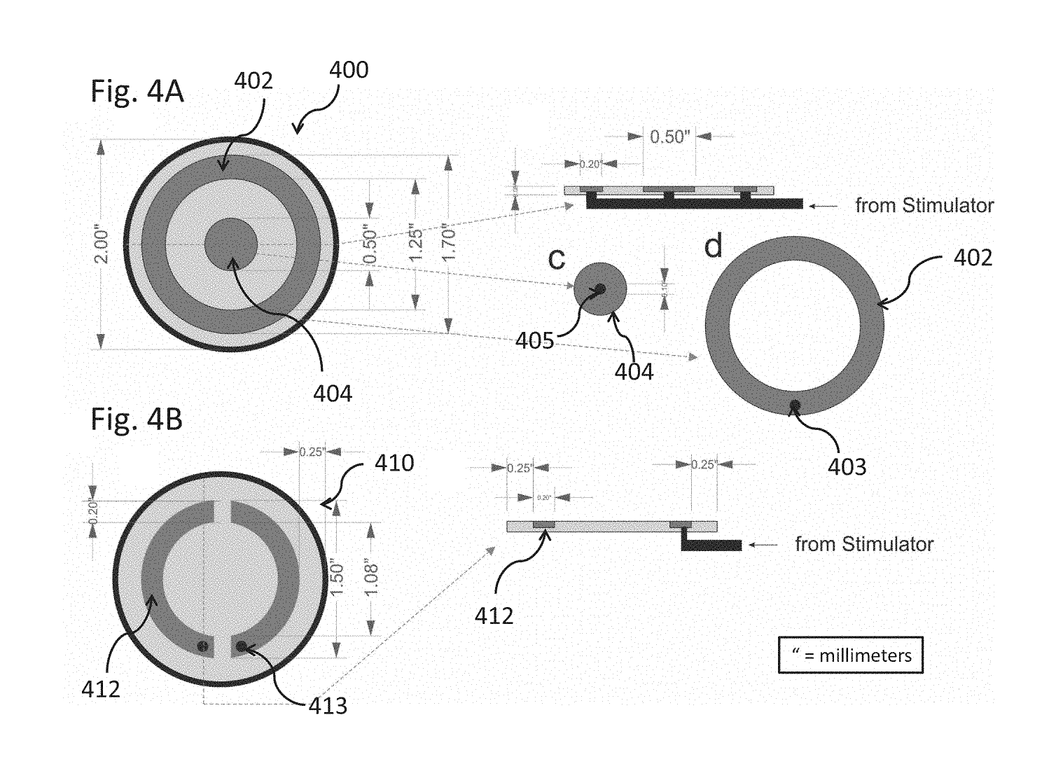

In some embodiments, the electric field is applied using a single electrode having a diameter in the range of 100 to 800 μm, for example 300, 400, 500 μm or any intermediate or larger diameter and a ring electrode having a width of at least 20 μm for example, 20, 40, 60, 80, 100 μm or any intermediated or larger value, for example the electrode arrangement shown in FIG. 4A. In some embodiments, an external ring electrode or an external arc electrode is associated with the external compartment. In some embodiments, the internal electrode and the external ring or arc electrode form a cathode-anode arrangement. Optionally, the internal electrode and the ring or arc electrode are connected to the same electrical output, for example to the same stimulator output, which optionally delivers a monophasic capacitor coupled stimulation. In some embodiments, the central electrode diameter is in a range of 100-1000 μm, for example 100-300 μm, 200-500 μm or 400-600 μm, for example 500 μm. In some embodiments, the central electrode is circular, ellipsoid or rectangular.

In some embodiments, the ring or arc electrode surrounds the internal electrode. In some embodiments, the width or the distance between the inner circumference and the outer circumference of the ring is in a range of 1-300 μm, for example 1-100 μm, 50-200 μm or 100-400 μm. In some embodiments, the ring electrode is circular, ellipsoid or rectangular.

In some embodiments, the internal and/or the external electrodes are embedded into the floor as an electrode foil soldered with the wires bundled into a common stimulation wire, for example as shown in FIG. 4A. In some embodiments, the wires, for example stimulation wires are soldered to the internal and external electrodes, for example as depicted on FIGS. 4A-4B.

In some embodiments, two arch-shaped electrodes, for example electrodes for application of an electric field, are assembled into the floor of the internal compartment, for example as shown in FIG. 4B. In some embodiments, electrodes for delivery of an electric field are made from platinum-iridium or stainless steel wires. In some embodiments, the electrodes are arranged to maximize the even distribution of the applied electrical field, for example electric current, over the whole surface of neurons in the internal compartment. In some embodiments, electrodes positioned in the internal compartment are used for application of an electric field. Alternatively, the electrodes are used for measuring electric properties of the cells, for example by switching from the output terminals of the stimulating device to the input terminals of the measuring device. In some embodiments, the switching could be done with the variety of commutating devices, for example a simple switch, electromechanical or solid-state relay, multiplexor, etc. In some embodiments, the switching device depends on the specific design choice. In some embodiments, the stimulator's design will be based on a StimDuino stimulator as published in DOI:10.1016/j.jneumeth.2015.01.016.

In some embodiments, the electrode associated with the internal compartment is configured to measure the electric properties of cells cultured in the internal compartment, for example neurons. In some embodiments, an electrode associated with the microgrooves is configured to measure the electric properties of the axons or the neurites extending from the neurons. Optionally, the electric properties of the axons or neurites are measured following the electric field application to the neurons. In some embodiments, the electric properties of the muscle cells are measured following the application of the electric field to the neurons.

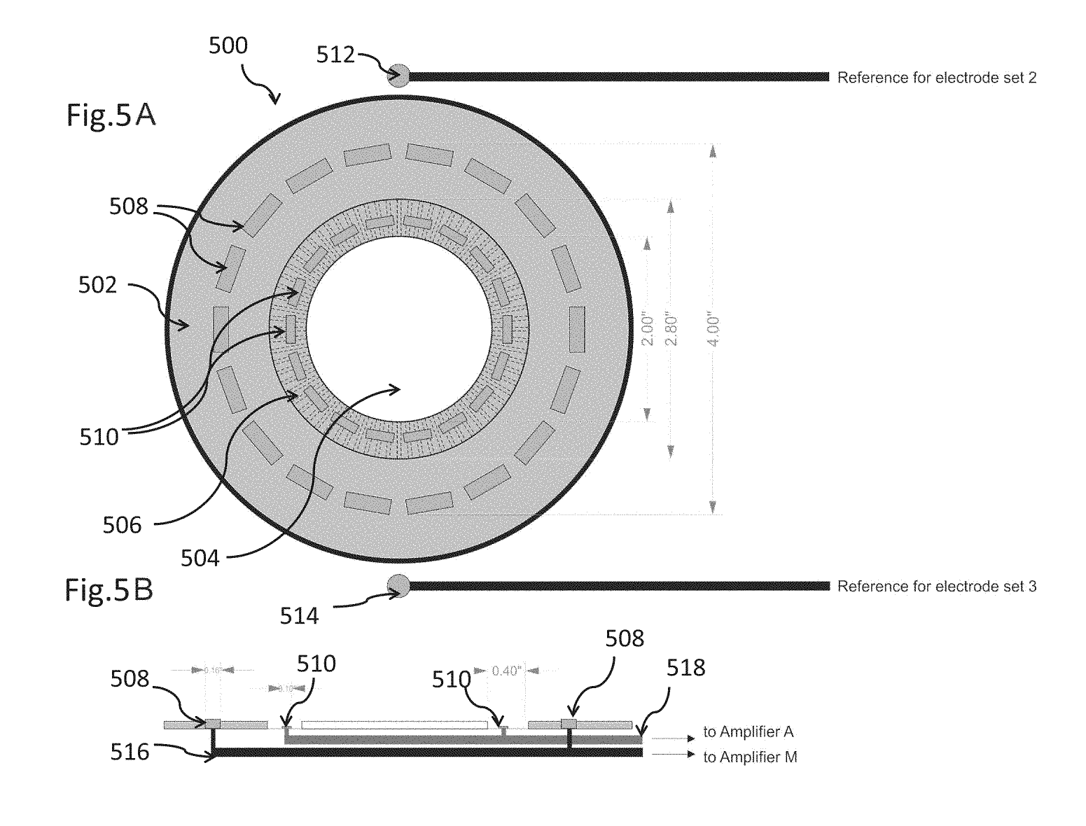

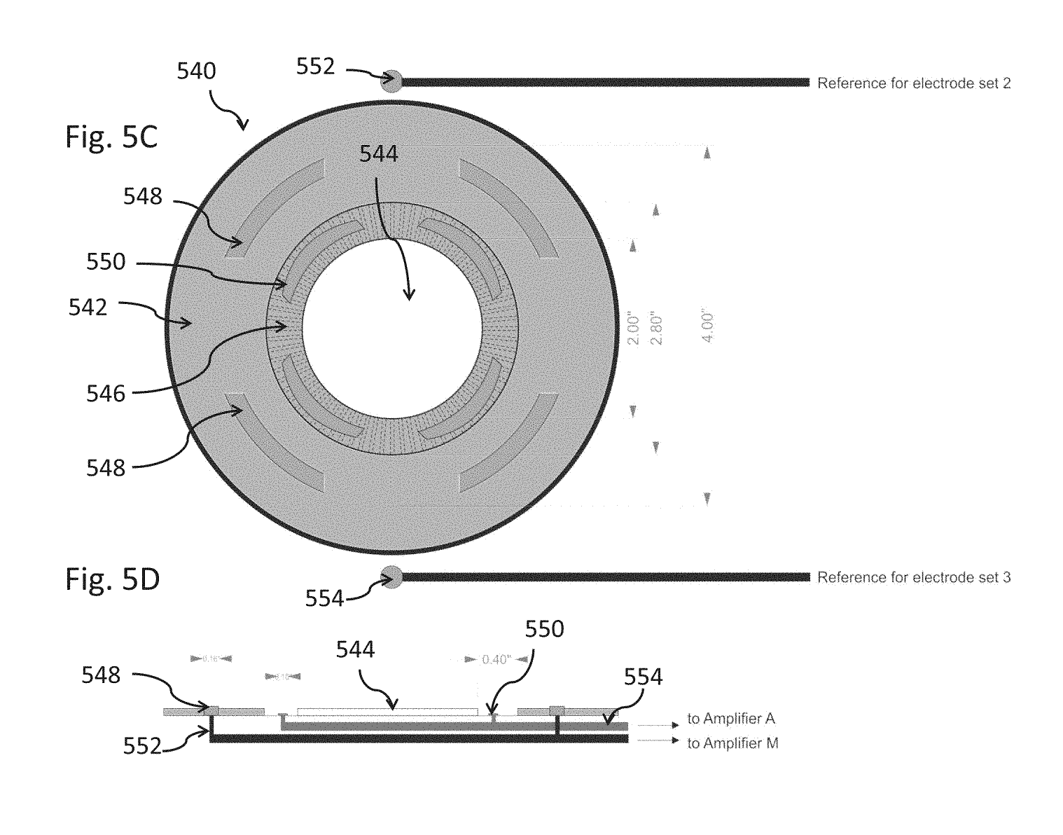

In some embodiments, the measuring or recording electrodes are connected via separate amplifiers. In some embodiments, ring electrodes either split into a plurality of serially arranged electrodes, for example as shown in FIGS. 5A and 5C or are shaped as an arc or are shaped as continuous ring electrodes, for example as shown in FIG. 4A. In some embodiments, the measuring electrodes are located under the groove guiding axons from the neurons in the internal compartment towards the target cells (muscles or neurons) in the external compartment (electrode set #3, or recording electrode of “A” amplifier). In some embodiments, the width or the distance between the inner circumference and the outer circumference of the ring electrodes is in a range of 5-500 μm, for example 100 to 200 μm, 200-300 μm or 10-150 μm or any intermediate range or range of distances.

In some embodiments, the base layer of the external compartment contains embedded recording electrodes. In some embodiments, the electrodes in the external compartment are arranged as at least two segments of ring electrodes, for example as shown in FIGS. 5A and 5C, or shaped as at least two arches or shaped as a continuous ring electrode (as shown in FIGS. 4A and 4B). In some embodiments, each arch of the arch electrodes has an angle of at least 20 degrees, for example 20 degrees, 45 degrees, 90 degrees or 180 degrees or any intermediate or larger angle. In some embodiments, the width or the distance between the inner circumference and the outer circumference of the ring electrodes is in a range of 5-500 μm, for example 100 to 200 μm, 200-300 μm or 10-150 μm or any intermediate range or range of distances (electrode set #2 or recording electrode of “M” amplifier). In some embodiments, at least two ring electrodes are associated with the external compartment for example, to increase sensitivity and resolution of data acquisition (electrode set #2 and 2a, wired to amplifiers “M” and “Ma”; not shown on FIG. 5A)

In some embodiments, electrodes associated with the microgrooves and the external compartment are embedded into the base layer as foils of platinum-iridium or stainless steel soldered to the wires connecting the electrodes to two different amplifier sets (A—amplifier set for compound action potential measurement from the intercompartment microgroove region (microgrooves) and M or Ma—amplifier sets for field potential change in the external compartment). In some embodiments, each recording electrode is paired with at least one reference electrode outside the compartmental chamber associated with a reference chamber.

In some embodiments, an electric field is applied to recording electrode set #3 associated with microgrooves between the internal and external compartments. In some embodiments, the electric field is delivered to the electrode as discussed above for example, by switching between a stimulator and an amplifier. In some embodiments, electric field application to the axons or neurites in the microgrooves is used for example, in cases of intensive stimulation protocols, required for neuron-neuron synapse functionality study to prevent metabolic exhausting of the presynaptic neurons.

In some embodiments, the microfluidic chamber is round. In some embodiments, the round microfluidic chamber is sized and shaped to be positioned in a cell culture plate. Optionally, the microfluidic chamber is sized and shaped to be positioned in a well of a culturing plate having at least 2 wells, for example 4 wells, 6 wells, 8 wells, or 12 wells or any intermediate or larger number of wells. Alternatively, the culturing plate is pre-manufactured with the microfluidic chamber.

In some embodiments, each of the electrodes per microfluidic chamber is electrically isolated from all the other electrodes. In some embodiments, each of the electrodes is connected by wiring to a computational unit. In some embodiments, the electrodes are connected via separate wires to a control circuitry of the computational unit. Optionally, the electrodes are connected to the control circuitry through an amplifier. In some embodiments, electrodes from 2 or more microfluidic chambers are connected to the same computational unit.

In some embodiments, the control circuitry measures the electric properties of the muscle or/and neuronal cells following the addition of at least one pharmaceutical or bioactive agent, to the culturing medium of the culturing compartment. In some embodiments, the electric properties of cells cultured in the microfluidic chamber are measured following a genetic manipulation of at least one cell type. Alternatively or additionally, the electric properties of cells cultured in the microfluidic chamber are measured following the addition of bioactive agents, for example short or long non-coding RNAs, microRNAs, siRNAs, anti-sense oligonucleotides and peptides.

In some embodiments, the control circuitry compares the muscle cells electric measurements of cells cultured in one microfluidic chamber, for example muscle cells to another microfluidic chamber, for example to determine the effect of the pharmaceutical or bioactive agent on the cultured cells. In some embodiments, the electric measurements are used to determine the effect of the pharmaceutical or bioactive agent on the functionality of neuromuscular junctions or neural synapses.

According to some embodiments, at least two electrodes are associated with muscle cells and/or myotubes in the external compartment. In some embodiments, at least one of the electrodes is associated with muscle cells and/or myotubes forming NMJ with axons, for example for recording the electrical activity of NMJ, and at least one different electrodes is associated with muscle cells and/or myotubes not forming NMJ, for example for recording a reference electrical signal. Optionally, the muscle cells and/or myotubes are cultured in a section of the external compartment not accessible to axons, for example in a section not connected to microgrooves or that the microgrooves are blocked. Alternatively, a reference signal is measured from a different microfluidic chamber that contain only muscle cells and/or myotubes without neuronal cells.

A possible advantage of culturing neuronal cells in a central chamber of a round microfluidic compartment and muscle cells in an external compartment, at least partly surrounding the central chamber is that it allows to increase the chances of NMJ formation by better aligning axons emanating from the neurons in the central chamber with muscle cells and/or myotubes in the external chamber.

An aspect of some embodiments of the invention relates to determining the effect of a pharmaceutical or a bioactive agent by measuring electrical properties from a plurality of cells, for example neurons, muscle cells, glia cells, or any cell type able to be part of a synapse. In some embodiments, the effect of the pharmaceutical or bioactive agent on the neuromuscular system or other synapses is determined. Optionally, the effect of the pharmaceutical or bioactive agent on the functionality neuromuscular junctions or other synapses is determined. In some embodiments, the effect of the pharmaceutical or bioactive agent on the generation of functional synapses, for example neuromuscular junctions is determined. In some embodiments, the effect of a genetic manipulation of at least one cell type which is part of a synapse is determined. Alternatively or additionally, the effect of bioactive agents, for example short or long non-coding RNAs, microRNAs, siRNAs, Anti-sense oligonucleotides and peptides is determined.

In some embodiments, the effect of the pharmaceutical or bioactive agent on synapses, for example neuromuscular systems is determined, for example by comparing electrical properties of two populations of muscle cells. Optionally, one of the muscle cells populations is used as a reference, for example, when one of the muscle cells populations is not treated with the pharmaceutical or bioactive agent.

In some embodiments, the electrical properties of a first cell population, for example muscle cells is measured following application of an electric field to a second cell population, for example motor neurons innervating the muscle cells. In some embodiments, the effect of the pharmaceutical or bioactive agent on the propagation of the electric signal between the two cell populations, for example from the neurons to the muscle cells is determined. Optionally, the effect of the pharmaceutical or bioactive agent on the propagation of the signal towards the first cell population, for example muscle cells is determined by measuring the electrical properties of axons innervating the muscle cells.

In some embodiments, the measured electrical property is an extracellular field potential. In some embodiments, the field potential is analyzed by optionally sequential procedures, including baseline subtraction, temporal alignment by stimulation artifact, peak detection will be performed, peak amplitude, phase, duration and kinetic parameters will calculated. Additionally or optionally, peak waveform is identified, delay between compound action potentials in axons and the target (muscle cells in some embodiment) is calculated, spatial coherence between neural stimulation and muscle cell compound action potential is evaluated. In addition to time domain, the waveforms are optionally assessed in frequency domain. In some embodiments, all measured parameters are clustered, optionally according to the specific synaptic functionality, using non-hierarchic clustering approach. In some embodiments, statistical reliability of clusters is assessed for each particular synaptic functionality. Optionally, synaptic functionality specific clusters are used for standardization of numerical values of synaptic functionality for diagnostic purposes.

According to some embodiments, the pharmaceutical or a bioactive agent is added to at least one external chamber, optionally at least one peripheral chamber, for example to allow measurements of the electrical activity of a cell population exposed to the pharmaceutical and/or to the bioactive agent compared to a cell population seeded in a different region of the microfluidic chamber that is not exposed to the pharmaceutical and/or to the bioactive agent.

According to some embodiments, the pharmaceutical or a bioactive agent is added to at least one external chamber which includes a lower amount of culturing media compared to an internal chamber of the microfluidic chamber. In some embodiments, a larger amount of culturing media within the internal media compared to the amount of culture media in the at least one external chamber prevents passage of the pharmaceutical or the bioactive agent into the internal chamber.

An aspect of some embodiments of the invention relates to a microfluidic chamber with at least three electrodes for measuring electric properties of at least two cell populations, for example muscle cells, neuronal cells or any other cell type capable of being part of a synapse. In some embodiments, at least one electrodes of the three electrodes measures electric properties of neuronal extensions. In some embodiments, the microfluidic chamber comprises at least two spaced-apart compartments for culturing at least two different cell types, for example neurons and muscle cells. In some embodiments, a plurality of microgrooves or channels connects the two spaced-apart compartments. In some embodiments, the microgrooves create a barrier that prevents fluid flow between the two compartments and therefore isolating the two compartments. In some embodiments, isolating each compartment allows for example, to exclusive treat one cell population or to provide a different treatment for each compartment. In some embodiments, providing an exclusive treatment to a specific compartment allows for example, to determine the effect of a drug on the various measurements whether it is applied only to cells cultures in one compartment, to cells cultured in a second compartment or to both compartments. In some embodiments, neuronal extensions from neurons cultured in one compartment penetrate through the microgrooves towards cells cultured in the second compartment, for example muscle cells, neurons, glia cells or any other cell type capable of being part of a synapse. In some embodiments, one of the three electrodes is associated with the microgrooves, for example for measuring the electric properties of the neuronal extensions. Additionally, a second electrode is associated with the second compartment for example, for measuring the electric properties of the neurons, glia or muscle cells.

In some embodiments, a third electrode of the three electrodes is associated with a compartment used for culturing neurons, for example for applying an electric field to the cultured neurons. Alternatively or additionally, the electrode measures the electric properties of the neurons, for example to determine the spontaneous electric activity of the neurons. In some embodiments, the electric properties of the muscle cells, glia cells or a second population of neurons are measured by a first electrode following application of an electric field to a first population of neurons by a second electrode.

In some embodiments, the device comprising a microfluidic chamber associated with the electrodes as described herein, that is used for the study of synaptic function. In some embodiments, the device is used for the study of interaction between two populations of neuronal cells, for example between two types of neurons. In some embodiments, at least one of the neuronal subpopulation comprises a heterogeneous primary neuronal culture. In some embodiments, the device is used to study neuron-glia interactions for example, to test the role of glia cells in central nervous system (CNS) functionality. In some embodiments, the device is used for the study of synaptic interactions between neuronal tissue and muscle tissue, as discussed herein.

In some embodiments, the device is used for the study of synaptic interactions between neuronal tissue and glandular tissue. In some embodiments, glandular tissue or cells, for example glandular epithelial cells are cultured in the external compartment. In some embodiments, the secretion ability of the glandular cells is studied for example, by tweaking of the external compartment for chemical indicator reaction for secretion identification. In some embodiments, the secretion ability of the glandular cells following electric filed application to neurons innervating the glandular cells is studied.

In some embodiments, the device, for example the microfluidic device comprises at least two spaced-apart compartments. In some embodiments, the compartments are fluidically isolated for example, to allow culturing of cells in different culturing mediums.

An aspect of some embodiments relates to aligning cultured cells, according to a micropattern on a surface within a tissue culture chamber. In some embodiments, the micro-patterned surface is a surface of at least one electrode found within the chamber. Alternatively or additionally, the micro-patterned surface is the base surface of the chamber. In some embodiments, a micro-patterned surface is a surface which includes a pattern suitable for grouping and/or alignment of cells.

According to some embodiments, the micro-patterned surface comprises at least one alignment element, for example a recess or channel shaped and sized for alignment of muscle cells. In some embodiments, the aligned muscle cells form at least one myotube within the alignment element. In some embodiments, the surface comprises a plurality of alignment elements, for example a plurality of recesses and/or channels, optionally aligned relative to each other. In some embodiments, a plurality of aligned elements used for culturing of muscle cells allows, for example to form a plurality of myotubes. Optionally the myotubes are aligned in parallel or in an angle relative to each other.

According to some embodiments, the micropatterned surface is a surface of a single electrode used for recording a single electrical signal corresponding to the electrical activity of a plurality of muscle cells or myotubes. Alternatively, the micropatterned surface comprises a plurality of electrodes, optionally used for recording of a plurality of electrical signals from one or more cultured muscle cells or myotubes.

A possible advantage of recording electrical signals from two or more myotubes aligned in parallel, is that the recorded signals do not mask each other, and are optionally summed to an amplified signal which allows, for example to increase signal to noise (SNR) ratio.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Exemplary Method for Screening the Effect of Pharmaceutical or Bioactive Agents on Synapse Function

According to some exemplary embodiments, cells are seeded in a microfluidic chamber. In some embodiments, neurons, for example, motor neurons and/or motor neuron progenitors are seeded in one compartment of the microfluidic chamber, optionally an internal compartment. Alternatively, spinal cord explants are seeded in the central compartment. In some embodiments, stem cells, for example induced pluripotent stem cells (iPSC) are seeded in the internal compartment. In some embodiments, muscle cells or myotubes are seeded in a different compartment of the microfluidic chamber, optionally an arc compartment of at least 180 degrees, for example 180, 270, 360 degrees or any intermediate or larger angle. In some embodiments, the arc compartment surrounds the central compartment. In some embodiments, any type of cells capable of being part of a synapse are seeded in a second compartment of the microfluidic chamber, for example glandular cells, glia cells or a different population of neurons.

According to some exemplary embodiments, the bottom surface of the compartments used for seeding the cells, for example spinal cord explants and/or the motor neurons and/or the muscle cells or any other type of cells are coated by at least one organic material, for example, to increase cell the adhesion of the cells to the bottom surface. In some embodiments, the organic material used for the coating is selected from a list of laminin, fibronectin, poly-1-lysine, poly-1-ornithine, or matrigel or any other type of material capable of increasing the adherence of the cells to the bottom surface of the compartments.

According to some exemplary embodiments, the seeded cells are cultured in a culturing medium for a desired time period. In some embodiments, the culturing medium comprises at least one neurotrophic factor, for example brain-derived neurotrophic factor (BDNF), nerve growth factor (NGF), neurotrophic factor 3 (NT-3), and/or neurotrophic factor 4 (NT-4), Glial derived neurotrophic factor (GDNF), and/or Ciliary neurotrophic factor (CNTF) or any other factor capable of increasing the proliferation and/or differentiation rates of the cells. In some embodiments, the neurons are cultured until neurites start to emanate from the cell bodies of the neurons and to make contact with the muscle cells. In some embodiments, the neurons are cultured for a desired period that allows, for example, formation of NMJ between the neurites and the muscle cells. In some embodiments, the desired period is in a range of 1-21 days, for example 3, 4, 5, 6, 7, 8 days. In some embodiments, the neurites penetrate through a plurality of microgrooves connecting the neurons chamber with the muscle cells chamber. Optionally, the neurites penetrate through a plurality of microgrooves connecting the internal compartment with the external compartment, for example arc compartment. In some embodiments, some of the neurites differentiate into axons that penetrate through the microgrooves towards the muscle cells in the arc compartment.

According to some exemplary embodiments, a pharmaceutical or a bioactive agent is added to the cell culture medium. In some embodiments, a pharmaceutical or a bioactive agent is added to at least one of the cultured cells, for example muscle cells. Alternatively or additionally, the pharmaceutical or bioactive agent is added to the neurons. In some embodiments, the pharmaceutical or bioactive agent is slowly released into the culturing medium during a treatment period.

According to some exemplary embodiments, an electric field is delivered to the neurons. In some embodiments, the electric field is applied to a plurality of cell bodies of the neurons. In some embodiments, the electric field is applied by an electrode placed in a substrate layer of the neurons chamber. In some embodiments, the electric field is applied by an electrode placed in the substrate layer of the central chamber. In some embodiments, the electrode is in direct contact with a plurality of neurons, optionally with the cell bodies of the neurons.

According to some exemplary embodiments, the electric properties of the neurites are measured, optionally after the application of the electric field. In some embodiments, the electric properties of the neurites are measured by an electrode, for example an arc electrode, a ring electrode or any type of segmented electrode. In some embodiments, the arc electrode is an arc of at least 20 degrees. In some embodiments, the electrode is positioned in the substrate layer of the microgrooves. Optionally, the electrode is in a direct contact with the neurites.

According to some exemplary embodiments, the electric properties of cells, for example, the muscle cells are measured by a different electrode, optionally an arc electrode or a ring electrode. In some embodiments, the arc electrode is shaped as an arc of at least 180 degrees, for example 180, 270, 300 or any intermediate or larger angle. In some embodiments, the electric properties of the cells, for example the muscle cells are measured following the electric field application. Alternatively, the electric properties of the cells are measured before the electric field application. In some embodiments, the electrode measures the electric properties of a plurality of muscle cells, or any type of cells cultured in the microfluidic chamber. In some embodiments, the electrode is positioned in the substrate layer of the external compartment, optionally in the substrate layer of the external ring or arc compartment. In some embodiments, the electrode is in a direct contact with cells cultured in the external compartment.

According the some exemplary embodiments, the measured electric properties of the cells cultured in the external compartment, for example muscle cells and/or of the neurites found in the microgrooves are compared to reference measurements, for example reference measurements from the external compartment and/or from the microgrooves. In some embodiments, the reference measurements are of cells, for example muscle cells and/or neurites that are not exposed to the pharmaceutical or bioactive agent. In some embodiments, the reference measurements are of cells, for example muscle cells and/or neurites that are exposed to a different concentration of the pharmaceutical or bioactive agent. In some embodiments, the reference measurements are of cells, for example muscle cells and/or neurites that are exposed to the pharmaceutical or bioactive agent for different periods. In some embodiments, the reference measurements are of cells, for example muscle cells and/or neurites prior to the application of the electric field to the neurons.

According to some exemplary embodiments, the effect of the pharmaceutical or bioactive agent is determined based on the measured electric properties. In some embodiments, the effect of the pharmaceutical or bioactive agent on the functionality of synapses, for example on the functionality of NMJ is determined based on the electric properties measurement of the muscle cells and/or neurites. In some embodiments, the effect of the pharmaceutical or bioactive agent on the generation of functional synapses, for example on the generation of NMJ is determined based on the electric properties measurement of the muscle cells and/or neurites. In some embodiments, the effect of the pharmaceutical or bioactive agent on the number of functional synapses, for example on the number of functional NMJ compared to non-functional synapses or NMJ is determined based on the electric properties measurement of the cells, for example muscle cells and/or neurites.

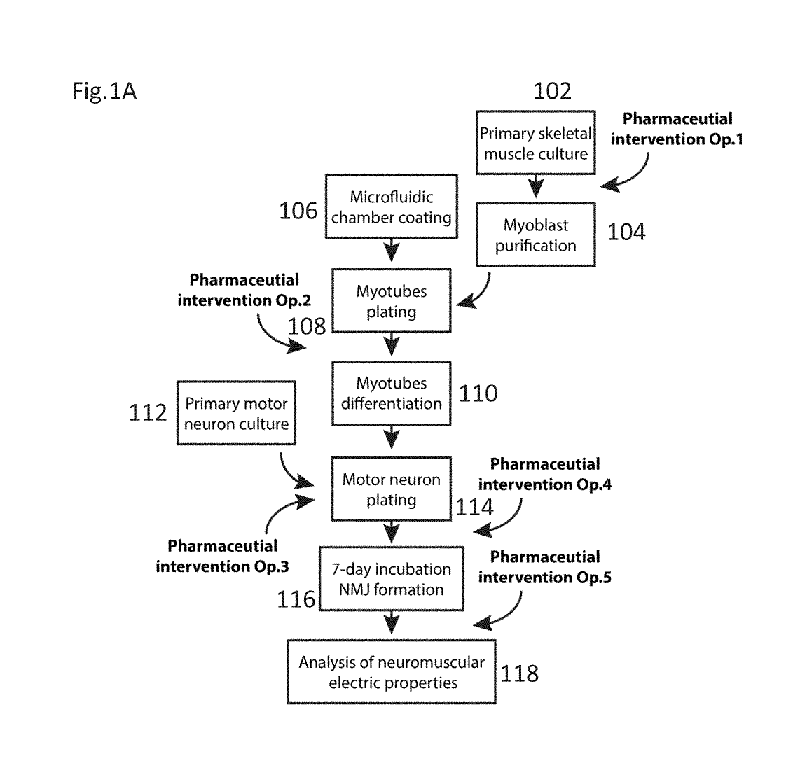

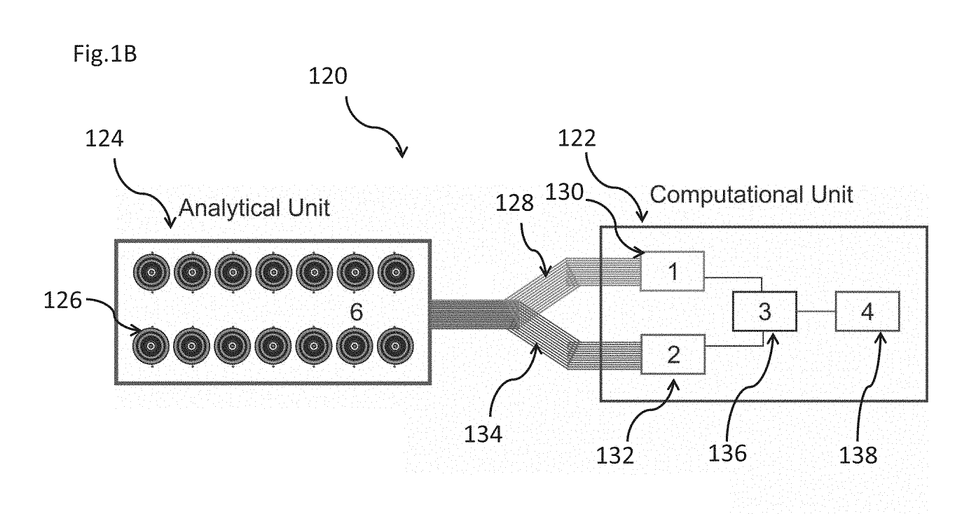

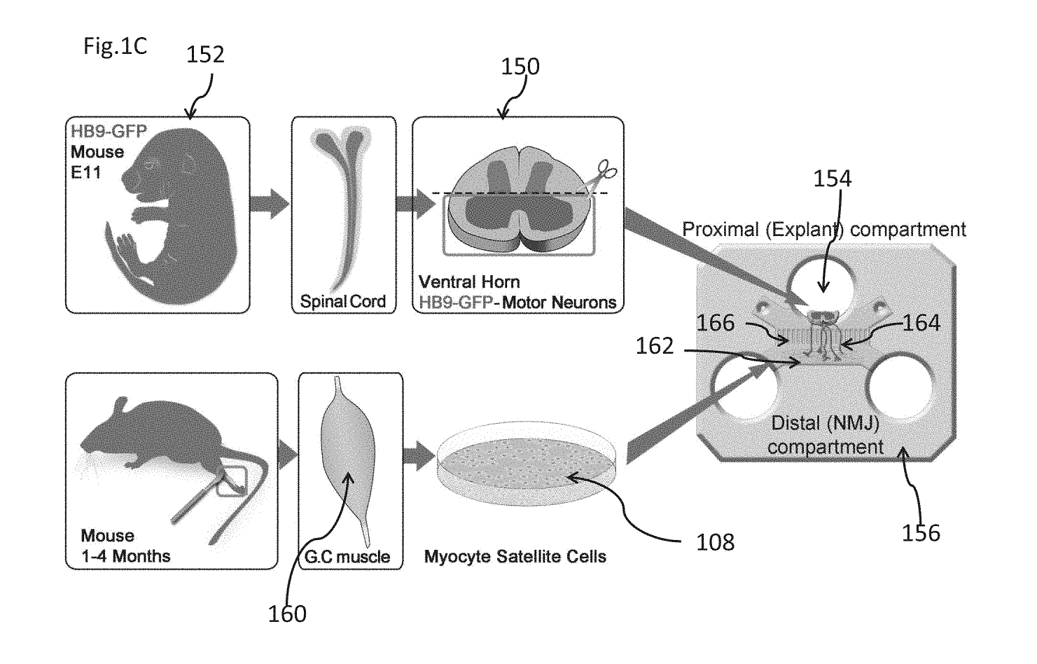

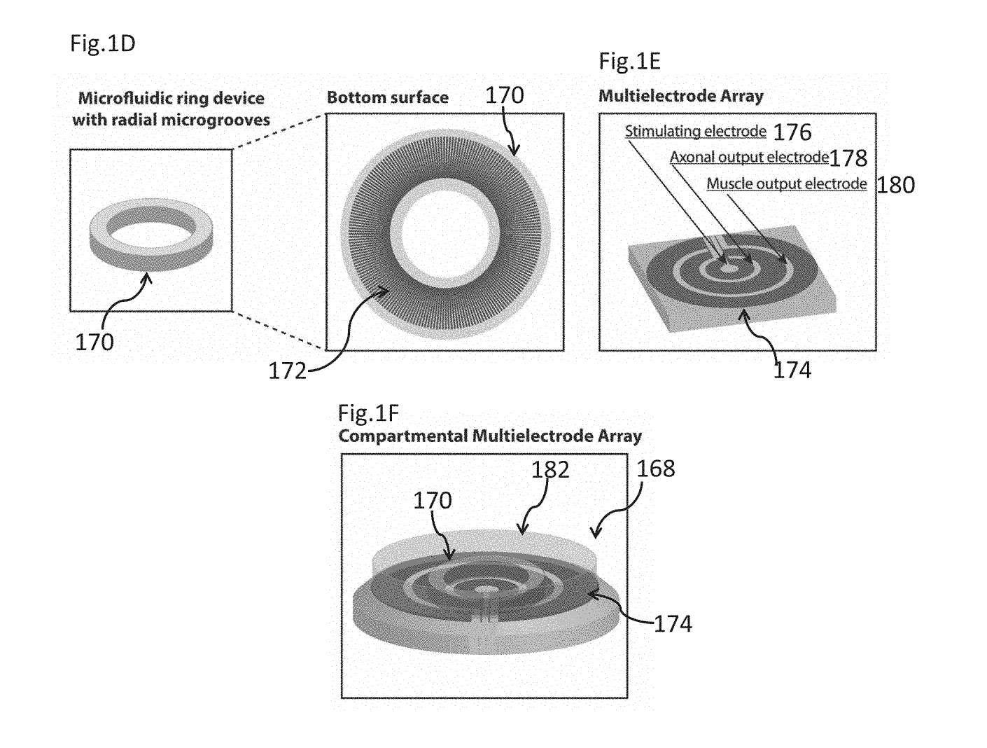

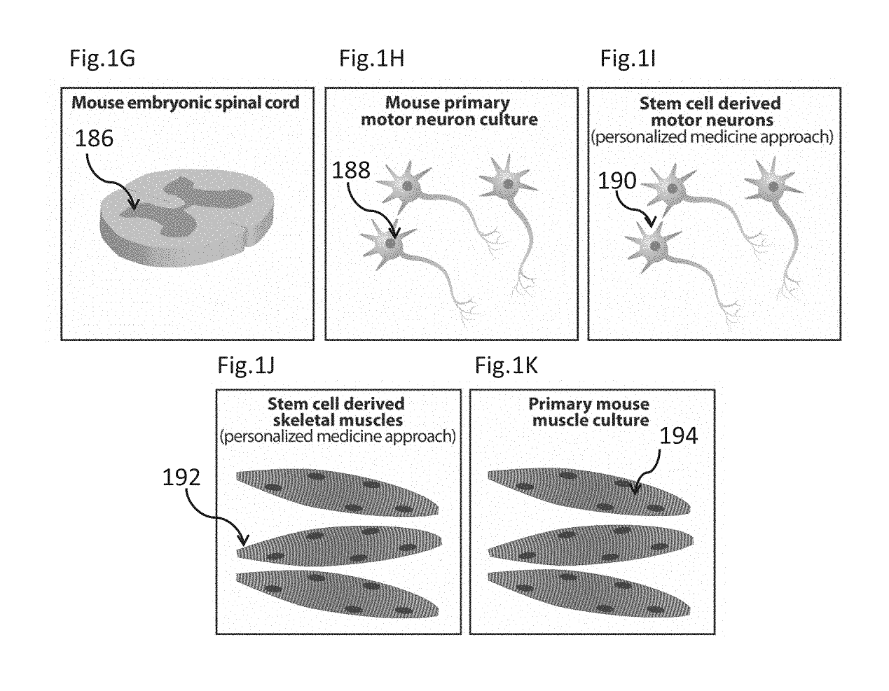

Reference is now made to FIG. 1A, depicting a method for screening the effect of a pharmaceutical or a bioactive agent or a biological intervention on NMJ, according to some embodiments of the invention.

According to some exemplary embodiments, a primary skeletal muscle is generated and cultured at 102. In some embodiments, primary myoblast cells are purified from mouse skeletal muscle at 104. In some embodiments, a pharmaceutical or a bioactive agent is provided to the primary skeletal muscle culture at 102 or to the purified myoblasts at 104.

According to some exemplary embodiments, at least one compartment of the microfluidic chamber is coated at 106. In some embodiments, the compartment is coated with a coating material comprising laminin, fibronectin, poly-1-lysine, or poly-1-ornithine or any other compound that is suitable to increase the adherence of cells to the compartment. In some embodiments, the purified myoblasts are plated at 108. Optionally, the purified myoblasts or myotubes are plated in the coated compartment.

According to some exemplary embodiments, the myotubes are differentiating at 110. In some embodiments, a pharmaceutical or a bioactive agent is provided to the plated myotubes or to the differentiated myotubes.

According to some exemplary embodiments, a primary culture of motor neurons is prepared at 112. In some embodiments, the motor neurons are plated into a different compartment of the microfluidic chamber at 114. In some embodiments, a pharmaceutical or a bioactive agent is provided to the plated motor neurons.

According to some exemplary embodiments, the plated motor neurons and the myotubes are cultured for at least 1 day, for example 2, 3, 4, 5, 6, 7, 8, 9, 10, 11, 12, 13, 14 days or any intermediate or larger time period at 116. In some embodiments, the motor neurons and the myotubes are cultured for a sufficient time period for example, to allow generation of NMJ between the neurons and the myotubes. In some embodiments, a pharmaceutical or a bioactive agent is provided to the neurons and/or to the myotubes during NMJ generation.

According to some exemplary embodiments, electric properties of the cultured cells are measured at 118. In some embodiments, electric properties of the NMJ are measured for example, to determine the functionality of the NMJ.

Exemplary System for Measuring Electric Properties of NMJ

According to some exemplary embodiments, a system for measuring the electric properties of synapses, for example NMJ, comprises an analytical unit and a computational unit.

According to some exemplary embodiments, an analytical unit comprises a microfluidic chamber of at least two compartments for example, grooves, shaped and sized for culturing of cells. In some embodiments, the microfluidic chamber is round and sized to fit into a single cell culturing plate. In some embodiments, the microfluidic chamber is sized to fit into a well of a culturing plate, for example into a culturing plate of at least two wells, for example a culturing plate of 4, 6, 8, 12 wells or any intermediate or larger number of wells.

According to some exemplary embodiments, one of the compartments is an internal compartment, and a second compartment is an external compartment, for example an external ring compartment. In some embodiments, the external compartment is an arc compartment with an angle of at least 180 degrees, for example 180, 270, or 300 degrees or any intermediate or larger angle. In some embodiments, the external compartment surrounds the internal compartment. Optionally, the external and internal compartments are concentric or coaxial. In some embodiments, the external and internal compartments are connected by a plurality of microgrooves, for example channels. In some embodiments, the microgrooves width allows penetration of neurites or axons but not of neuronal cell bodies.

According to some exemplary embodiments, the internal compartment is optionally a round compartment. In some embodiments, the internal compartment is sized and shaped for seeding and culturing of stem cells, neurons or neuronal explants, for example a spinal cord explant, or motor neuron progenitors. In some embodiments, the external compartment, for example arc or ring compartments are sized and shaped for seeding and culturing of neurons, muscle cells, muscle progenitors or any cell type capable of being part of a synapse.

According to some exemplary embodiments, the base layer of the microfluidic chamber comprises at least three electrodes' sets. In some embodiments, one of the electrodes, for example an internal electrode is positioned in the base layer of the internal compartment. In some embodiments, the electrode of the internal compartment is configured to deliver an electric field to cells cultured in the internal compartment, for example to neurons or spinal cord explant cells, or to motor neurons. In some embodiments, the electrode of the internal compartment is configured to measure the electric properties of cells cultured in the internal compartment, for example neurons, spinal cord explant cells or motor neurons. In some embodiments, the electrode is in a direct contact with the cells cultured in the internal compartment. In some embodiments, the first electrode is positioned under the cells, for example as shown in FIG. 4A. In some embodiments, the size of the first electrode is smaller or similar to the size of the internal compartment. In some embodiments, the diameter of the first electrode is smaller or similar to the diameter of the internal compartment.

According to some exemplary embodiments, a third electrode or electrode set is positioned in the base layer of the external compartment, for example the arc compartment or the ring compartment. In some embodiments, the third electrode is configured to measure the electric properties of target cells, for example muscle cells, myotubes or neuronal cells cultured in the compartment. In some embodiments, the electrode is configured to apply and/or acquire an electric field or electrical field potential alteration in the cells cultured in the external compartment, for example the arc or the ring compartment, optionally in response to stimulation or an electric field applied to the internal compartment. In some embodiments, the electrode is positioned under the cells.

According to some exemplary embodiments, the second electrode is shaped as a ring or an arc with an angle of at least 180 degrees, for example 180, 200, 270, 300 degrees or any intermediate or larger angle or as serially positioned segments of circular electrode. In some embodiments, the width of the second electrode is smaller or approximates to the width of the arc or ring compartment. Alternatively, additional ring or arc electrodes are associated with the arc or ring compartment for example, for better sensitivity and resolution coverage. In some embodiments, the ring electrode diameter is smaller or similar to the diameter of the arc compartment. In some embodiments, the second electrode surrounds the first electrode. In some embodiments, the second electrode and the first electrode are concentric or axial.

According to some exemplary embodiments, a third electrode or electrode set is positioned in the substrate layer of the microgrooves connecting the two compartments. In some embodiments, the third electrode is configured to measure the electric properties of neuronal processes, for example neurites or axons in the microgrooves. Alternatively or additionally, the third electrode is configured to apply an electric field to the neuronal processes. In some embodiments, the third electrode is an arc electrode with an angle of at least 180 degrees for example, 200, 270, 300 degrees or any intermediate or larger angle. Alternatively, the third electrode is a ring electrode. In some embodiments, the third electrode surrounds the first electrode. In some embodiments, the third electrode and the first electrode are concentric or axial. In some embodiments, the size of the third electrode is smaller or similar to the size of the microgrooves. In some embodiments, the width of the third electrode is similar to the width of the microgrooves. In some embodiments, the diameter of the third electrode is smaller or similar to the diameter of the microgrooves. In some embodiments, the third electrode is positioned between the first electrode and the second electrode. In some embodiments, the third electrode is positioned in a shorter distance from the first electrode compared to the distance between the first electrode and the second electrode.

In some embodiments, the diameter and width of the electrode change during an optimization process; they depend on electrical field distribution geometry and evenness of this distribution. In some embodiments, the electrodes diameter is in a range of 50-500 μm for example, 20-150 μm, 100-300 μm, 400-500 μm or any intermediate size or range of sizes.

According to some exemplary embodiments, the electrodes of the microfluidic chamber are connected to the computational unit through separate wiring. In some embodiments, the electrodes are connected to a control circuitry of the computational unit. In some embodiments, the electrodes are connected to a stimulating device circuitry commanded from the software of the computational unit. In some embodiments, the electrodes are connected to an amplifier included in the computational unit, for example as shown in FIG. 1B.

In some embodiments, the computational unit comprises an electric field generator, for example a stimulus generator. In some embodiments, the control circuitry signals the electric field generator to generate an electric field. In some embodiments, the electric field is delivered through one of the electrodes of the analytical unit to the cells. In some embodiments, the computational unit comprises a memory storage for example, for storing log files of the system and/or electric field parameters and/or electric field protocols. In some embodiments, the control circuitry measures electric properties of the cells using at least one electrode of the analytical unit. In some embodiments, the control circuitry stores the measured electric properties in the memory storage. In some embodiments, the control circuitry analyses the measured electric properties according to at least one program stored in the memory storage.

In some embodiments, the computational unit comprises an interface. In some embodiments, the interface comprises a display. In some embodiments, the control circuitry displays the measured electric properties and/or the analysis results on the display. In some embodiments, the computational unit receives input from a user via the interface. In some embodiments, the interface delivers a human detectable indication, for example a sound indication and/or a light indication to the user.

Reference is now made to FIG. 1B depicting a system for measuring electric properties of synapses, according to some embodiments of the invention.

According to some exemplary embodiments, a synapse analysis device 120 comprises an analytical unit 124 and a computational unit 122. In some embodiments, the analytical unit 124 comprises at least one microfluidic chamber, for example 1, 2, 4, 6, 8, 10, 12, 14 or any intermediate or larger number of microfluidic chambers. In some embodiments, each of the microfluidic chambers comprises an electrode array of at least 3 electrodes for example, for measuring the electric properties of cultured cells.

According to some exemplary embodiments, the computational unit 122 comprises a pulse generator, for example stimulus generator 130, also indicated as box 1 in FIG. 1B, for example for generating an electric field. In some embodiments, the pulse generator is electrically connected to some electrodes of analytical unit 124 by wiring 128. Optionally, wiring 128 is connected to at least one electrode of analytical unit 124. In some embodiments, the computational unit 122 comprises an amplifier, for example differential amplifier 132, also indicated as box 2 in FIG. 1B. In some embodiments, the amplifier is electrically connected via wires 134 to some of the electrodes of analytical unit 124 for example, to allow measuring of electrical properties of cells cultured in the analytical unit. In some embodiments, each electrode of analytical unit 124 is connected by a separate and electrically isolated wire to the pulse generator and/or to the amplifier of the computational unit 122. Alternatively, some of the electrodes are electrically interconnected by a single wire to the computational unit 122.

According to some exemplary embodiments, the computational unit 122 comprises a control circuitry for example, control unit 136, also indicated as box 3 in FIG. 1B. In some embodiments, the control circuitry is electrically connected to the pulse generator and/or to the amplifier. In some embodiments, the control circuitry 136 signals the pulse generator 130 to generate an electric field. In some embodiments, the control circuitry 136 receives measured values from the amplifier 132, and optionally analyses the measured values, for example to reduce noise or to identify at least one pattern within the measured values. In some embodiments, the control circuitry 136 is electrically connected to workstation 138, also indicated as box 4 in FIG. 1B, which includes an interface. In some embodiments, the interface displays some of the analysis results performed by the control circuitry 136 to a user, optionally on a display. Additionally, the interface is configured to deliver a human detectable indication, for example a sound and/or a visible indication to a user of the device.

According to some exemplary embodiments, the computational unit 122 comprises a communication component, optionally a communication circuitry for example for transmitting analysis results and/or for transmitting measured electric parameter values to a remote computer and/or to a handheld device. Additionally, the communication component receives information, for example work protocols from a remote computer and/or from a handheld device.

Exemplary Compartmental Microfluidic Chamber for NMJ Functional Assays

Reference is now made to FIG. 1C depicting a co-culture preparation in a microfluidic chamber, according to some embodiments of the invention. According to some exemplary embodiments, a compartmental chamber system, segregates the soma from the axon from motor neurons culture, for example HB9-GFP spinal cord MN cultures. In some embodiments, spinal cord explants or motor neurons 150 are isolated from an embryonic mouse, for example embryonic day e11.5 HB9-GFP mouse 152 and placed within the proximal channel 154 of a microfluidic chamber 156. In some embodiments, skeletal myocytes 158 are obtained from a skeletal or a smooth muscle, for example the Gastrocnemius muscle 160 of an adult mouse and added to the distal channel 162 of the microfluidic chamber 156. In some embodiments, motor neurons axons, for example HB9-GFP axons 164 grow thorough grooves 166 connecting the distal and the proximal channels to innervate the cultured myocytes and create functional synapses.

Exemplary NMJ-MEA Device

Reference is now made to FIGS. 1D-1F depicting a microfluidic chamber, according to some exemplary embodiments of the invention.

According to some exemplary embodiments, a microfluidic chamber, for example microfluidic chamber 168 comprises a microfluidic ring 170 which includes a plurality of microgrooves 172, optionally in the base layer of the ring 170. In some embodiments, each of the plurality of microgrooves is shaped and sized to allow passage of neuronal extensions, for example axons but not the passage of neuronal cell bodies. In some embodiments, the width of each of the microgrooves is up to 10 μm (micron), for example 1 micron, 5 micron, 10 micron or any intermediate, smaller or larger value.

According to some exemplary embodiments, the microfluidic chamber 168 comprises an electrode array, for example array 174 which includes at least one electrode. In some embodiments, the array 174 includes at least one central electrode, for example a stimulating electrode 176 for delivery of an electric field to a plurality of cells, for example plurality of cells of a first cell type. In some embodiments, the array 174 includes at least one additional electrode, for example electrode 180, at least partly surrounding the central electrode 176. In some embodiments, the electrode 180, optionally a ring electrode is used to record the electrical activity of a plurality of cells of a second cell type, for example muscle cells. Alternatively or additionally, the electrode 180 is used to deliver an electric field to the cells of the second cell type. In some embodiments, the electrode array 174 includes at least one electrode, optionally a ring electrode, for example an axonal output electrode 178 positioned between the central electrode 176 and the electrode 180. Optionally, the axonal output electrode 178 is used to record the electrical activity of axons. Alternatively or additionally, the axonal output electrode is used to deliver an electric field to the axons.

Reference is now made to FIGS. 1G-1K depicting optional cell types cultured in a microfluidic chamber, according to some exemplary embodiments of the invention. According to some exemplary embodiments, the microfluidic chamber, for example chamber 168 comprises at least one central compartment for culturing a first cell type and at least one peripheral compartment for culturing a second cell type.

According to some exemplary embodiments, the at least one central compartment is used for culturing at least one explant, for example a brain explant and/or a spinal cord explant, for example spinal cord explant 186 shown in FIG. 1G. Alternatively or additionally, a primary cell type, for example a mouse primary motor neuron cell type 188 shown in FIG. 1H is cultured in the central compartment. Alternatively, a human primary motor neuron cell type is cultured in the central compartment. In some embodiments, stem cells and/or stem cell-derived motor neurons 190 shown in FIG. 1I are cultured in the central compartment. Optionally, the cells cultured in the central compartment are genetically modified cells, for example genetically modified by insertion of a gene for a fluorescence protein.

According to some exemplary embodiments, a second cell type is cultured in the at least one peripheral compartment of the microfluidic chamber. In some embodiments, the second cell type comprises muscle cells and/or myotubes. In some embodiments, the muscle cells cultured in the at least one peripheral compartment comprise skeletal muscles, for example primary skeletal muscles or stem cell derived skeletal muscles 192 shown in FIG. 1J. Alternatively or additionally, the muscle cells cultured in the at least one peripheral compartment comprise primary mouse muscle cells 194, shown in FIG. 1K.

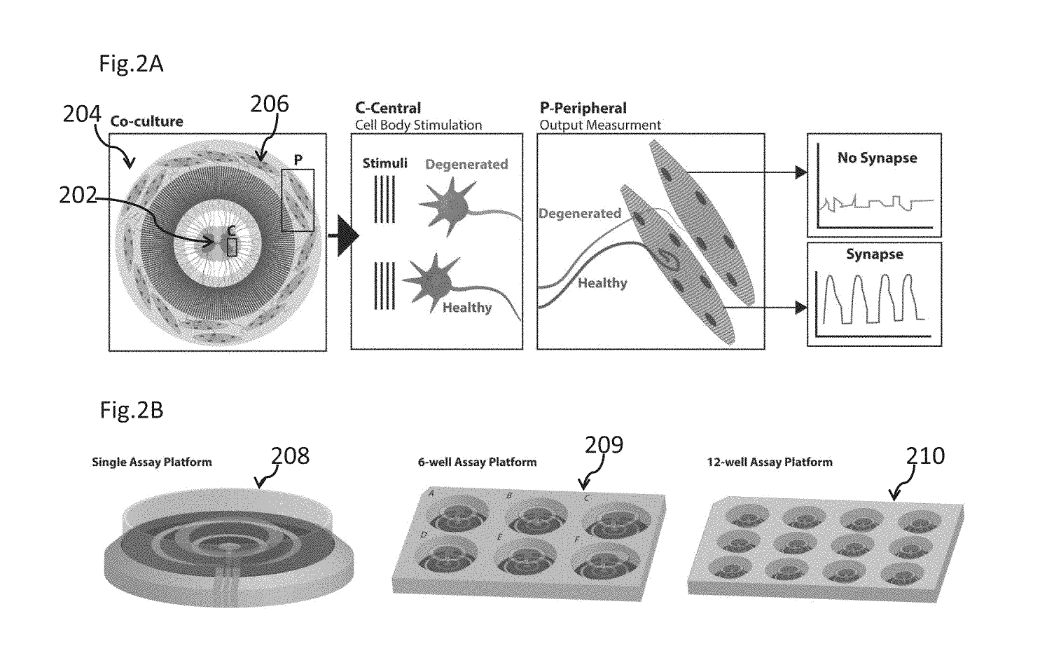

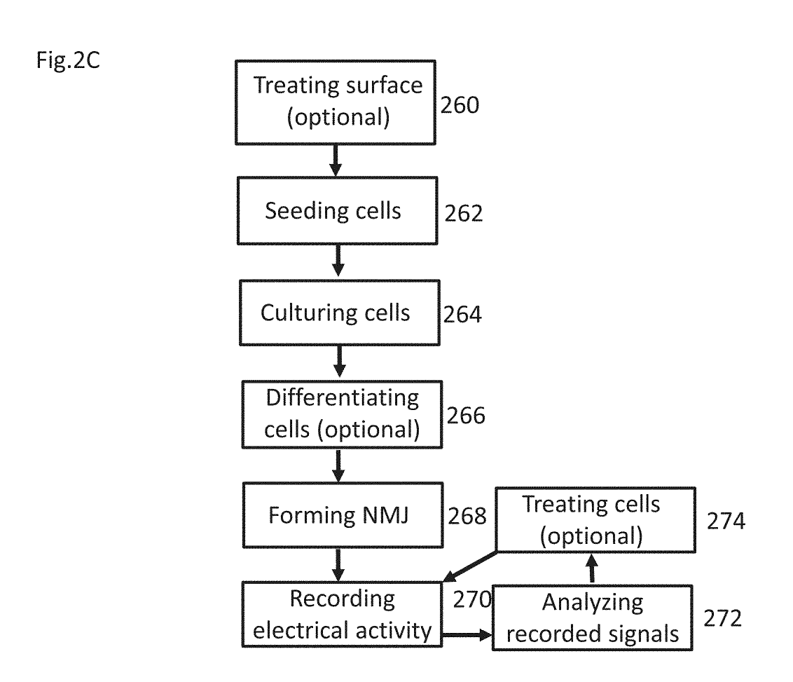

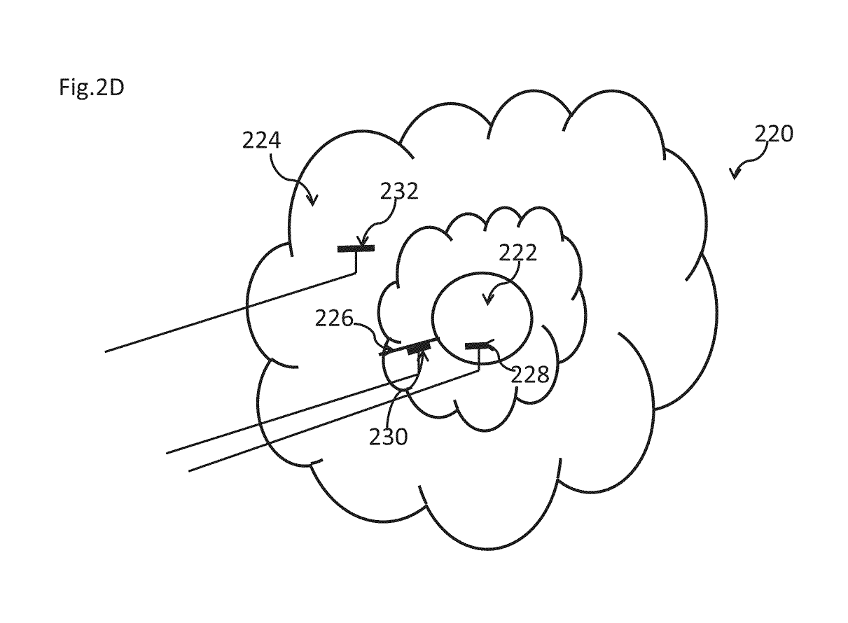

A potential advantage of the microfluidic chamber, for example the device is the capability to screen pharmaceutical or bioactive agents' effects on synapse function under normal and pathological situations. In some embodiments, the device hybridizes compartmental microfluidic and multi-electrode arrays (MEA). Reference is now made to FIGS. 2A-2B depicting a microfluidic chamber, for example an NMJ-MEA device according to some embodiments of the invention.

According to some exemplary embodiments, a spinal cord explant, for example spinal cord explant 202 is plated on the center of a microfluidic compartmental ring chamber, for example compartmental chamber 204 (presynaptic), muscle cells, for example muscle cells 206 are plated around the explant on the other side of the compartmental chamber 204 (post-synaptic). Alternatively, motor neurons derived from induced pluripotent stem cells (iPSC) are plated on the center of the microfluidic compartmental ring chamber. In some embodiments, neurons of the spinal cord explant 202, or the iPSC-derived motor neurons extend their axons via microgrooves (axonal region) into the post-synaptic chamber to innervate the muscle cells 206, for example to form functional in-vitro Neuromuscular Junctions (NMJs), mimicking in vivo NMJ behavior.

According to some exemplary embodiments, stimulating electrodes built into the presynaptic chamber base layer are used to trigger neuronal activity of the neurons. In some embodiments, the compartmental chamber comprises at least two recording-electrodes systems or electrodes sets. In some embodiments, one of the two recording-electrodes system is built within the axonal region bottom, for example to evaluate presynaptic activity in response to stimulation. In some embodiments, the second recording-electrodes system is built under the post-synaptic chamber, for example to evaluate the synapse functionality. In some embodiments, the synapse functionality is based on muscle cells electrical activity in response to the stimulation. In some embodiments, three electrode zones will be assembled on the plate bottom. In some embodiments, stimulation electrodes stimulate motor neurons, for example at the central chamber. In some embodiments, recording electrodes acquire signals from axons emanating from the motor neurons, for example, axons positioned at the microgrooves or channels. In some embodiments, recording electrodes acquire signals from muscle cells, for example, muscle cells positioned at a peripheral chamber. In some embodiments, each chamber is assembled into plates that comprise a plurality of wells and comprises electrical input and/or output for stimulation and/or recording.

According to some exemplary embodiments, an analytical module comprises at least two multi-compartmental chambers, for example 2, 3, 4, 5, 6, 10, 12 and any intermediate or larger number of chambers. In some embodiments, an analytical module comprises a single compartmental chamber, for example compartmental chamber 208. According to some exemplary embodiments, the analytical module comprises 6 multi-compartmental chambers, for example module 209 or 12 multi-compartmental chambers, for example module 210.

In some embodiments, each multicompartmental chamber is connected to a computational module, comprising a differential amplifier, a stimulator, an analog-to-digital converter and/or a computer-on-chip optionally with MATLAB-based data analysis software. A potential advantage of the computational module is that it provides the analyzed electrophysiological data not requiring electrophysiology expert as an operator. In some embodiments, the analytical module is disposable/recyclable.

Exemplary Culturing and Recording Process

According to some exemplary embodiments, at least two different cell types are cultured within at least two different culturing chambers of an analytical unit, interconnected by one or more channels. In some embodiments, the electrical activity of at least one of the cell types is recorded. Reference is made to FIG. 2C depicting a process for recording the electrical activity of at least two different cell types, which are optionally interacting, according to some exemplary embodiments of the invention.

According to some exemplary embodiments, an internal surface of at least one culturing chamber of an analytical unit is treated at 260. In some embodiments, the internal surface is treated, for example to increase cell attachment and/or cell differentiation. In some embodiments, the internal surface is treated, or example by coating the surface with one or more of Poly-L-Lysine, Poly-D-Lysine, Poly-Ornithine, Laminin, collagen, fibronectin

According to some exemplary embodiments, cells are seeded into at least one of the culturing chambers at 262. In some embodiments, neuronal cells and/or at least one neuronal explant, for example a spinal cord explant are seeded within a central chamber of the analytical unit. In some embodiments, muscle cells are seeded into one or more peripheral chambers, surrounding at least partly the central chamber.

According to some exemplary embodiments, seeded cells are cultured at 224. In some embodiments, the seeded cells are cultured in the same culturing medium. Alternatively, each of the cell types is cultured in a different cell medium.

According to some exemplary embodiments, at least one of the cultured cell types differentiates at 266. In some embodiments, the cells are differentiated in response to addition of at least one differentiating factor added to the culture medium. Alternatively or additionally, the cells differentiate in response to at least one differentiating factor released from the coating material and/or in response to the coating material, for example the coating material used for treating at least one of the culturing chambers at 260. In some embodiments, neuronal processes, for example axons, emanate from the neuronal cells and/or at least one neuronal explant seeded for example in the central chamber in response to brain-derived neurotrophic factor (BDNF), glial-derived neurotrophic factor (GDNF), insulin growth factor (IGF) and/or serum. Additionally or alternatively, muscle cells cultured in the at least one peripheral chamber fuse to form one or more myotubes over time and/or in response to poor media, for example DMEM culture media or Bioamf-2 culture media.

According to some exemplary embodiments, Neuromuscular junctions (NMJ) are formed at 268. In some embodiments, NMJ are formed by interaction between axons emanating from neuronal cells cultured in one chamber, optionally the central chamber, to muscle cells or myotubes cultured in at least one different chamber, for example at least one peripheral chamber.

According to some exemplary embodiments, the electrical activity of at least one cultured cell type is recorded at 270. In some embodiments, the electrical activity of the cultured neuronal cells is recorded, optionally by at least one electrode positioned within the central chamber. Alternatively or additionally, the electrical activity of muscle cells and/or myotubes is recorded by at least one electrode positioned in the at least one peripheral chamber. Optionally, the electrical activity of the axons travelling from the central chamber to the at least one peripheral chamber through one or more microgrooves is recorded by at least one electrode positioned within the microgrooves. In some embodiments, at least one electrode positioned within the central chamber is used to stimulate the neuronal cells in the central chamber. Optionally, at least one electrode positioned within the at least one peripheral chamber is configured to record the electrical activity of one or more muscle cells or myotubes in response to the stimulation.

According to some exemplary embodiments, the recorded electrical signals are analyzed at 272. In some embodiments, the recorded electrical signals are analyzed, for example to identify changes in electrical activity of muscle cells and/o myotubes in response to different bioactive agents, for example pharmaceutical drugs, chemicals, proteins, antibodies affecting NMJ. Alternatively or additionally, the recorded electrical signals are analyzed, for example to identify changes in electrical activity of muscle cells and/or myotubes when the cells are genetically manipulated.

According to some exemplary embodiments, at least one cell type is treated at 274. In some embodiments, cells, for example neural cells seeded and cultured in the central compartment are treated, for example by one or more drugs affecting axons growth, electric propagation through axons and/or formation of NMJ. Alternatively or additionally, muscle cells and/or myotubes cultured in the one or more peripheral chambers are treated. In some embodiments, the muscle cells and/or myotubes are treated with one or more drugs affecting myotubes formation, NMJ formation and/or electrical propagation within formed NMJ.

Exemplary Analytical Unit

According to some exemplary embodiments, in order to measure electrical activity of one or more cell types or of the interaction between two or more cell types, for example NMJ formed between axons and muscle cells, an analytical unit is used. Reference is now made to FIG. 2D depicting a block diagram of an analytical unit, according to some exemplary embodiments of the device.

According to some exemplary embodiments, the analytical unit, for example analytical unit 220 comprises at least two chambers, for example chamber 222 and chamber 224 for culturing at least two types of cells. In some embodiments, the two chambers are interconnected by at least one microgroove, for example channel 226. Additionally, the analytical unit comprises at least one electrode array configured to record electrical signals from at least one of the chambers 222, 222 and/or from the at least one microgroove 226.

According to some exemplary embodiments, one of the chambers is a central chamber, for example chamber 222. Optionally, the central chamber is shaped and sized for culturing neuronal cells and/or at least one neuronal explant, for example at least one spinal cord explant. In some embodiments, at least one of the chambers of the analytical unit is a peripheral chamber 224 for culturing muscle cells or myotubes. In some embodiments, the central chamber and the peripheral chambers are spaced apart, for example to prevent migration of intact cells from one chamber to another. In some embodiments, the microgrooves interconnecting the central and the peripheral chambers are shaped and sized to allow passage of axons emanating from the neuronal cell bodies cultured in the central chamber to the at least one peripheral chamber 224.

According to some exemplary embodiments, the analytical unit 220 comprises at least one electrode 228 positioned within the central chamber 222, configured to record electrical activity of neuronal cell bodies cultured in the central chamber 222, which are optionally at least partially in contact with the electrode 228. Additionally or alternatively, the analytical unit 220 comprises at least one electrode within the at least one peripheral chamber 224, for example for recording the electrical activity of muscle cells or myotubes cultured in the chamber 224, which are optionally at least partially in contact with the electrode. In some embodiments, the analytical unit comprises at least one electrode positioned within one or more of the microgrooves, for example to record electrical activity of cell protrusions, for example axons extending through the microgrooves.

According to some exemplary embodiments, at least part of the central chamber 222 is micro-patterned with at least two grooves, for example to direct axons towards the microgrooves 266. Alternatively, the microgrooves 266 extend at least partially into the central chamber. In some embodiments, the at least one peripheral chamber, for example chamber 224 is micro-patterned with at least one groove, for example to align muscle cells cultured in the peripheral chamber. Alternatively or additionally, an electrode positioned within the peripheral chamber, for example electrode 232 is a micro-patterned electrode with at least one groove, for example to align the muscle cells cultured in the peripheral chamber.

According to some exemplary embodiments, the at least one peripheral chamber 224 and/or the electrode 232 are micro-patterned with two or more grooves, optionally parallel grooves, shaped and sized for alignment of myotubes, optionally formed by fusion of muscle cells cultured within the grooves. In some embodiments, the two or more grooves are positioned, for example to allow alignment between adjacent myotubes and/or to allow alignment between axons and one or more of the myotubes.

According to some exemplary embodiments, an analytical unit of the device integrates a multielectrode system into a bicompartmental microfluidic chamber for example, to perform functional screening of NMJ or other types of synapses. In some embodiments, the compartmental chamber is shaped as a ring, for example to maximize the ability of the motor neurons to innervate as much as possible myotubes or other cells. In some embodiments, the internal and external compartments of the compartmental chamber are connected with microgrooves along the adjacent perimeters of the chamber.

According to some exemplary embodiments, microfluidic chambers are designed by casting polydimethylsiloxane (PDMS) onto silicon molds, which are optionally constructed by layering a channel-patterned layer, for example an 80-μm channel-patterned layer on top of a thick groove-patterned layer, for example 5-μm thick groove-patterned layer. In some embodiments, the thickness of the channel-patterned layer is at least 5 μm, for example 5, 10, 20, 30, 50, 80, 100 μm or any intermediate or larger value. In some embodiments, the thickness of the groove-patterned layer is at least 0.5 μm, for example 0.5, 1, 2, 3, 4, 5, 6, 8 μm or any intermediate or larger value.

In some embodiments, chamber molds are constructed through a photolithography process using a silicon (Si) wafer as a substrate, deposition of a 5 μm SU-8 photoresist layer that optionally forms the grooves and an 80-μm SU-8 photoresist layer that optionally forms the channels. Alternatively, the chambers and/or the molds are generated via 3D printing technology.

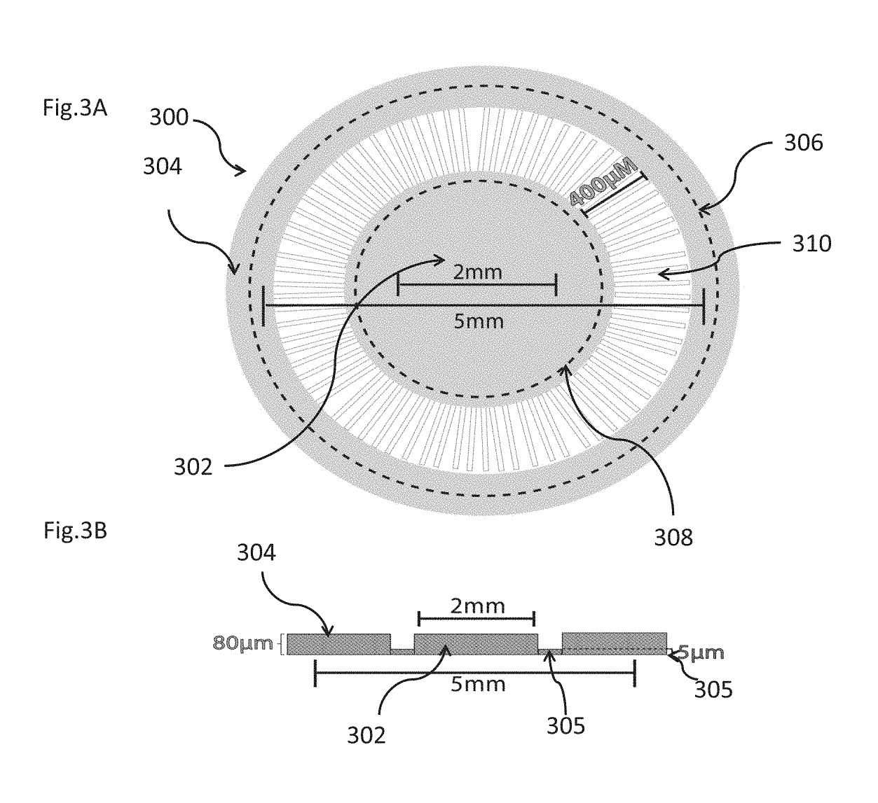

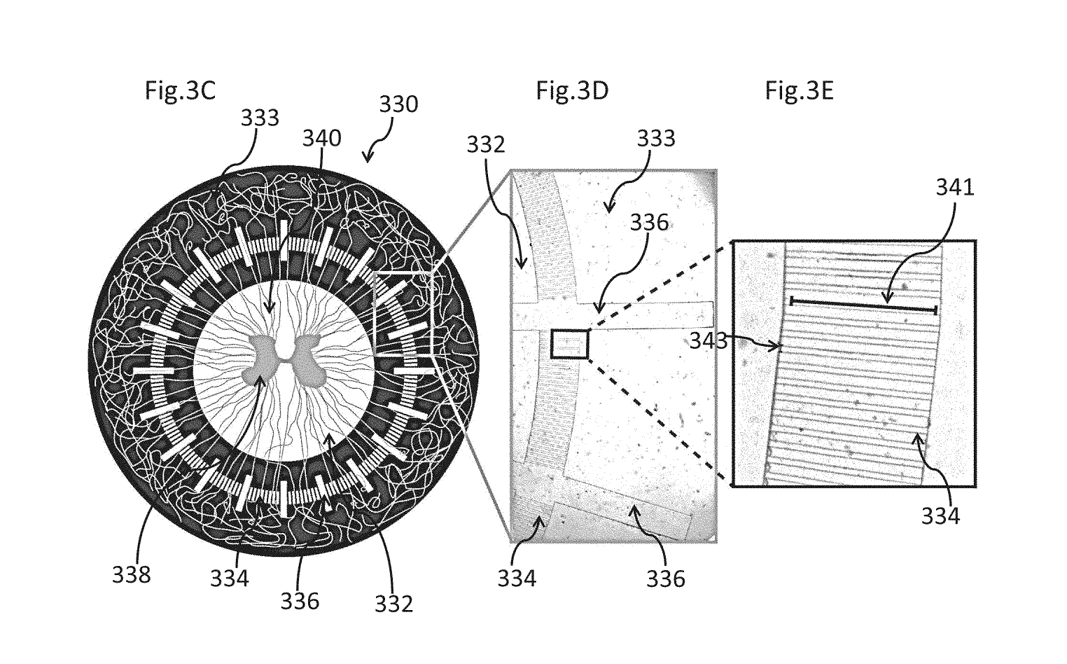

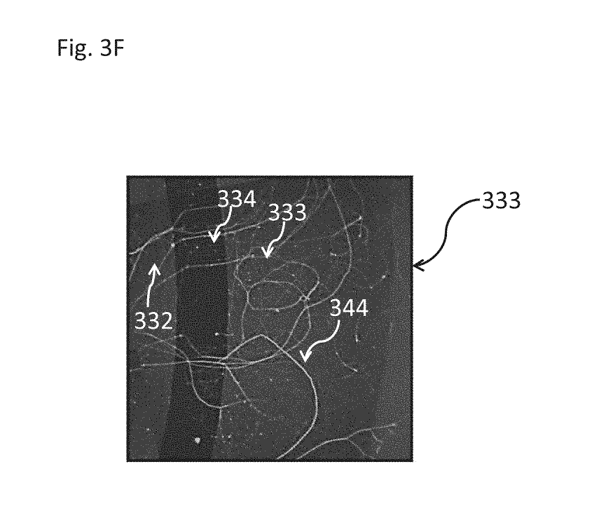

Reference is now made to FIGS. 3A-3B, depicting a compartmental chamber, according to some embodiments of the invention.

According to some exemplary embodiments, a microfluidic chamber, is combined with an electrode array, for example to form an analytical unit. In some embodiments, analytical unit 300 comprises an internal compartment 302, for example a central compartment, and at least one peripheral compartment, for example external compartment 304 for example, for culturing two different cell populations. In some embodiments, the diameter of the internal compartment is at least 0.5 μm, for example 0.5, 1, 2, 4, 5 μm or any intermediate or larger value.

In some embodiments, the external compartment is shaped as a ring. In some embodiments, the external compartment is shaped as an arc or comprises a plurality of arc-shaped segments with an angle of at least 10 degrees, for example 25, 45, 180 degrees. In some embodiments, a plurality of microgrooves positioned between the internal compartment 302 and the external compartment 304. In some embodiments, cells growing in the external compartment and/or in the internal compartment extend cell protrusions, for example neurites or axons through the microgrooves 310 into the other compartment. In some embodiments, the microgrooves form a barrier separating the internal and external compartment, for example to culture cells in two different cell culturing mediums. In some embodiments, the microgrooves length is at least 10 μm, for example 10, 20, 30, 100, 200, 300, 400, 500 μm or any intermediate or larger value.

According to some exemplary embodiments, the walls of the internal compartment and/or the walls of the external compartment have a height in a range of 10 μm to 200 μm, for example 10 μm, 20 μm, 50 μm, 80 μm, 90 μm or any intermediate, smaller or larger value. In some embodiments, the height of the microgrooves is in a range of 1 μm to 100 μm, for example 2 μm, 5 μm, 10 μm, 20 μm or any intermediate, smaller or larger value.

According to some exemplary, an electrode array is associated with the microfluidic chamber. In some embodiments, at least one electrode, for example electrode 306 is associated with the external compartment. In some embodiments, the electrode is positioned underneath the cells cultured in the external compartment. In some embodiments, the electrode 306 forms a continuous ring. Alternatively, the electrode 306 is one segment of a segmented electrode array associated with the external compartment. Optionally, electrode 306 is in a direct contact with some of the cells cultured in the external compartment.

According to some exemplary embodiments, at least one electrode is associated with the internal compartment, for example electrode 308. In some embodiments. In some embodiments, the electrode is positioned underneath the cells cultured in the internal compartment. Optionally, the electrode 306 is in a direct contact with some of the cells cultured in the internal compartment. In some embodiments, the electrode 306 aligned with an opening of one of the microgrooves for example for measuring or applying an electric field to a cell extension entering into the microgroove. In some embodiments, the electrode 306 is part of a segmented electrode array associated with the internal compartment. In some embodiments, the electrode forms a ring electrode.

According to some exemplary embodiments, the analytical unit of the microfluidic device, for example an NMJ-MEA device comprises three electrodes zones central zone electrodes are placed under the bottom of the central compartment and serve for example for placement of stimulation electrodes; inner ring zone electrodes are placed under the bottom of the microgrooves, for example for recording and/or assessment of motoneuron excitation; and the external ring electrodes are attached to the bottom of the external compartment, for example for recording of compound action potential generated by myotubes in response to neurotransmission across the NMJ. Exemplary neurons culturing in an analytical unit

According to some exemplary embodiments, neurons or at least one explant, for example a spinal cord explant are cultured in analytical unit. In some embodiments, the cultured neurons start to generate long neuronal processes, for example axons, that are directed towards a spaced apart culturing chamber through one or more microgrooves, for example channels. Optionally chemo-attractive compounds secreted from the spaced apart chamber attract the axons towards the chamber. Reference is now made to FIGS. 3C-3F depicting the growth of neurons in a first chamber of an analytical unit, and extension of axons through microgrooves into a second remote chamber, according to some exemplary embodiments.

According to some exemplary embodiments, for example as shown in FIG. 3C, a spinal cord explant 338 is cultured in a central chamber 332 of an analytical unit 330. In some embodiments, neuronal projections, for example axons 340 emanating from the explant 338 towards microgrooves 334. In some embodiments, one or more directing strips 336, are positioned between two microgroove sections, for example to direct the axons towards a microgroove section, optionally a selected microgroove section.

According to some exemplary embodiments, each of the microgrooves is shaped and sized to allow passage of one or more axons, but not cell bodies. In some embodiments, the largest dimension of each of the microgrooves openings, and/or the microgroove width 343 is in a range of 0.5-10 micron, for example 1 micron, 5 micron, 10 micron or any intermediate, smaller or larger values. In some embodiments, the length 341 of each microgroove, for example as shown in FIG. 3E is in a range of 1-500 micron, for example, 10 micron 100 micron, 300 micron, 400 micron, 500 micron or any intermediate, smaller or larger value.

According to some exemplary embodiments, for example as shown in FIG. 3F the axons 344 extend from a first culturing chamber, the central chamber 332 through the microgrooves into a second spaced apart chamber 333. In some embodiments, the axons within the second chamber, optionally a peripheral chamber, form arborized ends. In some embodiments, the second chamber 333, is shaped and sized for growing muscle cells, and for differentiation of muscle cells into myotubes. In some embodiments, the microgrooves prevent passage of large cell bodies with a diameter larger than 10 micron, but allow cell culture media to pass from one chamber to another.

According to some exemplary embodiments, the analytical unit comprises a radial ring-shaped microfluidic chamber layered over a culture dish surface. In some embodiments, the external diameter of the microfluidic ring, for example the ring of microgrooves 334 and the directing strips 335 is in a range of 6-20 mm, for example 5 mm, 9 mm, 15 mm or any intermediate, smaller or larger value. In some embodiments, the internal diameter of the microfluidic ring is in a range of 3-10 mm, for example 3 mm, 5 mm, 8 mm or any intermediate, smaller or larger value. (B) Light microscopy image of a region of the microfluidic ring. (C) Magnified image of the radial microgrooves. Dimensions of the microgrooves are specified. Groove length: 300 μm. Groove width: 10 μm. (D) Fluorescent image of motor axons (in green) extending through the microgrooves (black) from the central towards peripheral compartment.

Exemplary Computational Unit

According to some exemplary embodiments, a computational unit of an NMJ-MEA device comprises an amplifier, for example to amplify acquired signal obtained from the internal and external ring electrodes. In some embodiments, the signal is then analyzed by a software program, for example a MATLAB software program.

In some embodiments, the software program has one or more interfaces. In some embodiments, a basic interface of the program presents the outcome of automatically analyzed data to a person, who is not expert in electrophysiology. In some embodiments, an advanced interface of the program is used, for example for quality control, new protocol development and customization for use in research laboratories. A potential advantage of the computational unit is the ability to perform data analysis of a received signal as it is received and with minimal time delay.

In some embodiments, the analytical and the computational units are combined, optionally for functional screening of NMJ modeling pathological conditions, for example amyotrophic lateral sclerosis (ALS). In some embodiments, explants of the spinal cord and myoblasts of healthy mice and ALS mouse model, are plated and cultured in the compartmental chambers. In some embodiments, the cultured ALS cells are used, for example to evaluate functionality of the NMJ and/or tune the stimulation protocols and/or assemble libraries of field potential measurements for optimization of the automated data analysis. In some embodiments, pharmaceutical or bioactive agents, for example pharmaceutical agents known to affects motor neuron, muscle and NMJ activity are applied selectively to the compartmental chambers, for example to validate the quality of automated analysis.

Exemplary Electrode Array

Reference is now made to FIGS. 4A, 4B, 5A-5D depicting electrode arrays associated with a microfluidic chamber, according to some embodiments of the invention. It should be noted that in FIGS. 4A, 4B, 5A-5D the inches sign ″—denotes millimeters. According to some exemplary embodiments, for example as shown in FIG. 4A, an analytical unit 400 comprises a central internal electrode 404 and an external electrode, for example ring electrode 402 surrounding the internal electrode 404. In some embodiments, external electrode and the internal electrode are used for example, for applying an electric field to cells cultured in a microfluidic chamber. Alternatively, one of the electrodes is used for measuring the electric properties of the cells. In some embodiments, ring electrode 402 and internal electrode 404 are connected through the same wiring or through separate wiring to a pulse generator. In some embodiments, the wiring is connected through wiring connection 405 to the internal electrode 404. In some embodiments, the wiring is connected through wiring connection 403 to the ring electrode 402. In some embodiments, the internal electrode is associated with an internal compartment of the microfluidic chamber, that is optionally is used for culturing neuronal cells. In some embodiments, the electrode ring 402 is associated with microgrooves surrounding the internal compartment. In some embodiments, the electrode ring 402 is used for example, to deliver an electric field to neuronal extensions entering the microgrooves from the internal compartment. In some embodiments, the electrode ring 402 is used for example, to measure the electric properties of the neuronal extension, optionally following application of an electric field through the internal electrode.

According to some exemplary embodiments, for example as shown in FIG. 4B, an electrode comprises at least one arc electrode. In some embodiments, an analytical unit 410 comprises at least one arc electrode 412, for example 1, 2, 3, 4, 5, 6, 8 arc electrodes. In some embodiments, the arc electrode is associated with an external compartment of the microfluidic chamber, for example external compartment 304 in FIG. 3A. Alternatively, the arc electrode is associated with microgrooves connecting the external compartment and an internal compartment of the microfluidic chamber, for example microgrooves 310 in FIG. 3A. In some embodiments, electrical wiring from a pulse generator are connected to the arc electrode 412 through electrode connection 413. In some embodiments, each arc electrode is connected by a separate wiring to the pulse generator. Alternatively, some or all arc electrodes are connected by a shared wiring to the pulse generator.

Reference is now made to FIGS. 5A-5D, depicting a segmented electrode array, according to some embodiments of the invention.

According to some exemplary embodiments, an analytical unit 500 comprises two sets of segmented electrodes. In some embodiments, a set of segmented electrodes comprising at least two electrodes 508 and is associated with an external compartment of a microfluidic chamber, for example external compartment 502. In some embodiments, electrode 508 is configured to measure the electric properties of cells cultured in the external compartment. In some embodiments, a different set of electrodes, for example segmented electrodes 510 is associated with microgrooves 506 connecting an internal compartment 504 and the external compartment 502. In some embodiments, electrodes 510 are configured to measure the electric properties of neuronal extensions, for example axons penetrating through the microgrooves 506 into the external compartment 502.

In some embodiments, at least one reference electrode 512 is used for example as a reference electrode for electrodes 510. Additionally, a different reference electrode, for example at least one reference electrode 514 is used as a reference electrode to electrodes 508. In some embodiments, electrodes 508 and electrodes 510 are electrically connected by different electrical wiring to at least to different amplifiers. In some embodiments, electrodes 508 are electrically connected via electrical wiring 516 and electrodes 510 are electrically connected by electrical wiring 516 to different amplifiers.

Reference is now made to FIGS. 5C-5D, depicting a segmented electrode array, according to some embodiments of the invention.

According to some exemplary embodiments, an analytical unit 540 comprises two sets of segmented electrodes. In some embodiments, a set of segmented electrodes comprising at least four external electrodes 548 and is associated with an external compartment of a microfluidic chamber, for example external compartment 542. In some embodiments, electrode 548 is configured to measure the electric properties of cells cultured in the external compartment. In some embodiments, a different set of electrodes comprising at least four internal electrodes, for example internal electrodes 550 is associated with microgrooves 546 connecting an internal compartment 544 and the external compartment 542. In some embodiments, electrodes 546 are configured to measure the electric properties of neuronal extensions, for example axons penetrating through the microgrooves 546 into the external compartment 542.

In some embodiments, the external compartment 542 surrounds the internal compartment 544. In some embodiments, each of the external electrodes 548 faces an internal electrode of internal electrodes 550.

In some embodiments, at least one reference electrode 552 is used for example as a reference electrode for electrodes 548. Additionally, a different reference electrode, for example at least one reference electrode 554 is used as a reference electrode to electrodes 550. In some embodiments, external electrodes 548 and internal electrodes 550 are electrically connected by different electrical wiring to at least two different amplifiers. In some embodiments, external electrodes 548 are electrically connected via electrical wiring 552 and internal electrodes 550 are electrically connected by electrical wiring 554 to different amplifiers.

Exemplary NMJ Analysis Work Flow

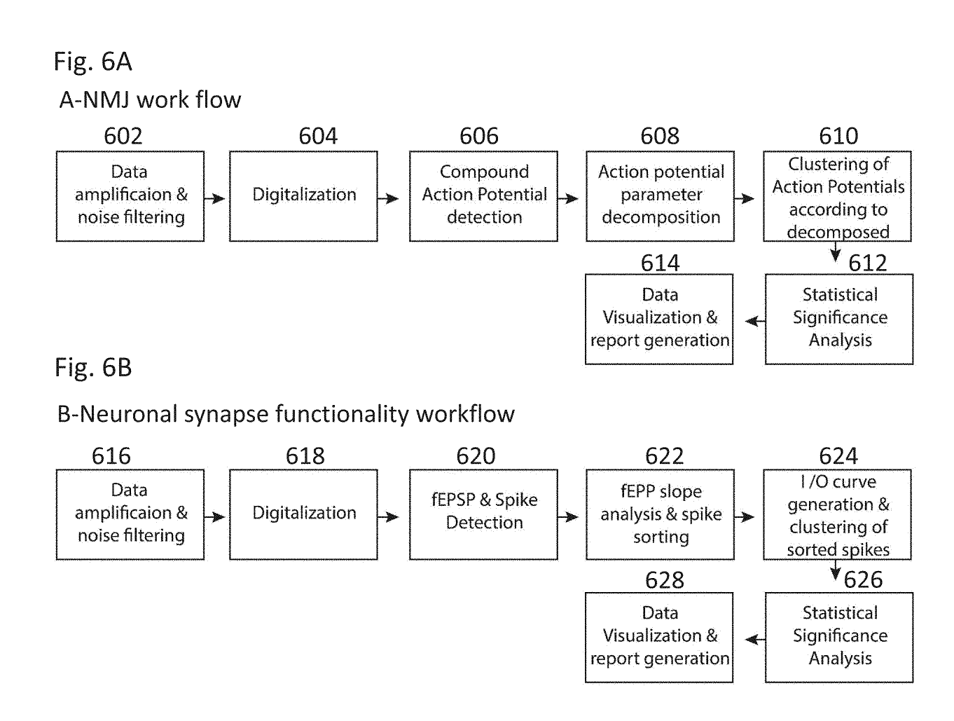

Reference is now made to FIG. 6A depicting a synapse analysis workflow, for example NMJ analysis workflow, according to some embodiments of the invention.

According to some exemplary embodiments, data is received and amplified at 602. In some embodiments, the data is filtered, for example to reduce noise or unrelated signals. In some embodiments, the data is digitized at 604. In some embodiments, a compound action potential is detected at 606, optionally based on the filtered signals. In some embodiments, action potential parameter is decomposed at 608. In some embodiments, action potentials are clustered at 610. In some embodiments, the action potentials are clustered according to the decomposition data. In some embodiments, a statistical analysis is performed at 612. In some embodiments, the data is visualized at 614. Optionally a report which comprises the data is generated.

Exemplary Neuronal Synapse Functionality Analysis Workflow

Reference is now made to FIG. 6B depicting a synapse functionality assay workflow, according to some embodiments of the invention.

According to some exemplary embodiments, data is received and amplified at 616. In some embodiments, the data is filtered, for example to reduce noise or unrelated signals. In some embodiments, the data is digitized at 618. In some embodiments, field excitatory postsynaptic potential (fEPSP) and spike are detected at 620. In some embodiments, the slope of the fEPSP is analysed at 622. Additionally or optionally spike are sorted. In some embodiments, an I/O curve is generated at 624. Additionally or optionally, sorted spikes are clustered at 624. In some embodiments, a statistical analysis is performed at 626, for example to determine statistical significance. In some embodiments, data following the statistical analysis is visualized. Additionally or optionally, a report comprising the data is generated.

Exemplary Method for Screening Drugs Capable of Restoring Synaptic Function

According to some exemplary embodiments, drugs, for example FDA-approved drugs are screened using the microfluidic device, to identify drugs capable of restoring synaptic function and/or prevent advancement of neurodegenerative diseases.

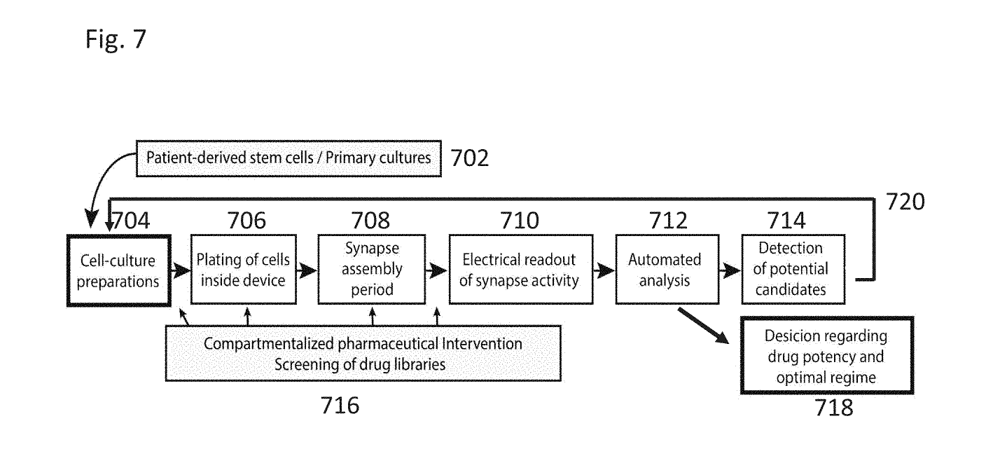

Reference is now made to FIG. 7, describing a process for screening drugs according to some embodiments of the invention.

According to some exemplary embodiments, primary cultures or patient-derived stem cells are obtained at 702. In some embodiments, the cells are cultured at 704. Optionally, the cells are prepared for plating inside the microfluidic device.

According to some exemplary embodiments, the cells are plated in the microfluidic device at 706. In some embodiments, following plating, cultures are grown for a period of time that allows the assembly of synapses between the cells in both compartments (peripheral and central) of the device at 708. In some embodiments, compartment-specific pharmaceutical interventions are applied at 716 for example at the time of plating, and/or during the synapse assembly period and/or immediately prior to electrical readout or following a challenge.

According to some exemplary embodiments, once pharmaceutical intervention period has ended, the electrical activity of cellular and synaptic circuits is recorded at 710. In some embodiments, the recorded electrical activity is analyzed, optionally automatically at 712. In some embodiments, the automated analysis software is capable of detecting samples in which the electrical activity deviates from the one of control and standard samples and supplies an output with a list of candidate drugs together with their analytical and statistical data at 714. In some embodiments, the candidate drugs are then trialed for the second time at 720 in order to test for various drug doses, administration regimes and combinations with other drug candidates in search for a synergistic activity and an optimized regime. In some embodiments, a decision regarding drug potency and/or a desired treatment regime is provided at 718. In some embodiments, at the end of a screen, we a report is supplied with our findings for potent FDA approved drugs and/or drug combinations that are able, in-our conditions, to restore proper circuit activity and may be beneficial to clinical therapy of the neurodegenerative disease tested.

### Exemplary Micro-Patterned Electrode

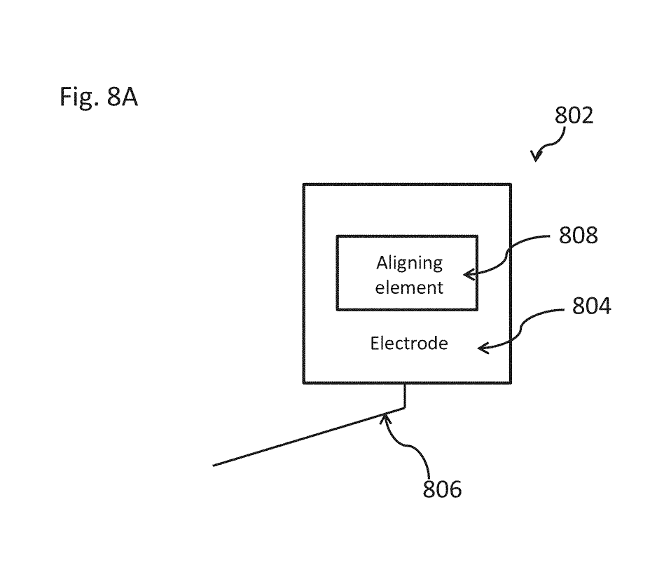

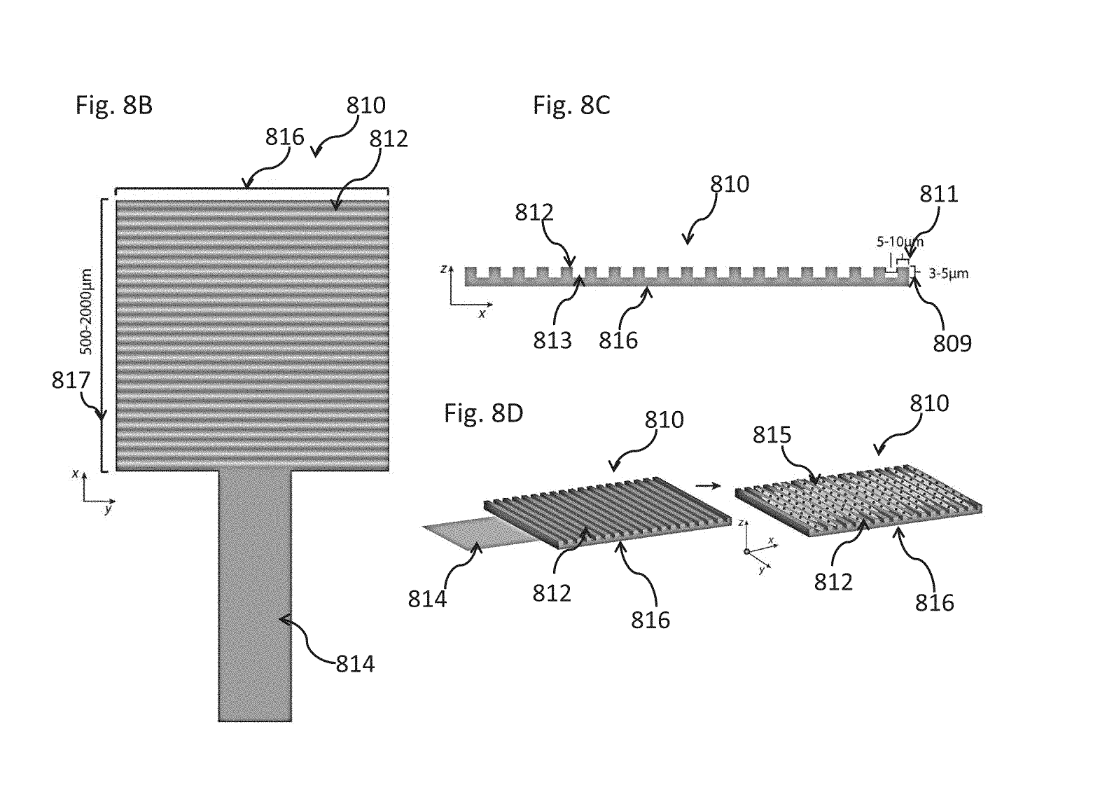

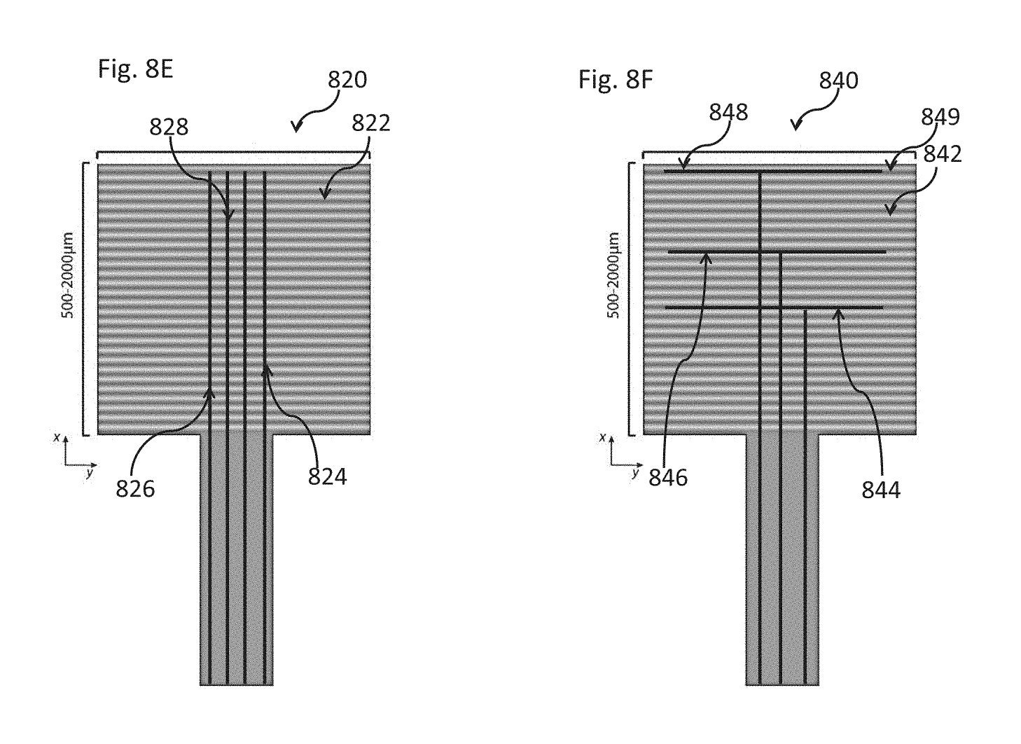

Reference is now made to FIG. 8A depicting a micro-patterned electrode, according to some exemplary embodiments.

According to some exemplary embodiments, a micro-patterned electrode 802 comprises an electrode 804 and at least one aligning element 808, for example a recess or a channel on top of the surface of the electrode 804. In some embodiments, the micro-patterned electrode comprises a plurality of aligning elements, which are optionally aligned in parallel relative to each other. Alternatively, the plurality of aligning elements are aligned in a selected angle relative to each other, for example in an angle of 5 degrees, 10 degrees, 30 degrees, 45 degrees, 90 degrees, 180 degrees or any other intermediate, smaller or larger angle.

According to some exemplary embodiments, the aligning element is shaped and sized for grouping and aligning cultured cells seeded on top of the electrode, for example aligning muscle cells. In some embodiments, the muscle cells are aligned within the aligning element to form at least one myotube, also termed herein as a muscle fiber. In some embodiments, the formed myotube has a length of at least 50 micron, for example 50, 100, 200 micron or any intermediate, smaller or larger value. In some embodiments, the aligning element 808 is used to form longer myotubes compared to non-aligned muscle cells. Alternatively and/or additionally, the aligning element 808 is used to form aligned myotubes which are aligned relative to each other, relative to the aligning element 808 and/or relative to nerve endings, for example axons.

According to some exemplary embodiments, the micro-patterned electrode comprises a plurality of aligning elements, optionally arranged in parallel to each other. In some embodiments, the aligning elements are shaped and sized to group cultured muscle cells to form a plurality of aligned myotubes, optionally a plurality of myotubes aligned in parallel relative to each other.

Reference is now made to FIGS. 8B-8D depicting a Micro-patterned electrode for the formation of aligned myotubes, for example muscle fibers in-culture.

According to some exemplary embodiments, for example as shown in FIG. 8B, a micro-patterned electrode 810 comprises at least one patterned surface, which comprises a plurality of aligning elements, for example stripes 812, optionally to generate a striated pattern. In some embodiments, the stripes 812 comprises one or more elongated recesses. In some embodiments, the electrode has a round shape, an oval shape, or a rectangular shape. In some embodiments, when the electrode is shaped as a rectangle, where the length of each side 816 or 817 of the electrode is in a range of 10-5000 micron. In some embodiments, the electrode comprises an electrical wiring 814 for example, for electrically connecting the electrode to an amplifier.

According to some exemplary embodiments, for example as shown in FIG. 8C, the height 809 of at least some of the stripes 812 is in a range of 1-10 micron, for example 1 micron, 3 micron, 5 micron or any intermediate, smaller or larger value. In some embodiments, the width of at least some of the stripes is in a range of 1-30 micron, for example 2 micron, 5 micron, 8 micron, 10 micron or any intermediate, smaller or larger value. In some embodiments, the width of at least some of the gaps 813 formed between two adjacent stripes is in a range of 1-30 micron, for example 2 micron, 5 micron, 8 micron, 10 micron or any intermediate, smaller or larger value. According to some exemplary embodiments, for example as shown in FIG. 8D, muscle cells 815 are seeded on top of the micro-patterned electrode 810 and are aligned within the gaps 813 or on top the stripes 815, for example to form aligned myotubes or aligned muscle fibers. In some embodiments, the entire micro-patterned electrode is electrically conductive. Alternatively or additionally, the stripes 812 and/or the gaps 813 are electrically conductive.

According to some exemplary embodiments, for example as shown in FIG. 8E, a micro-patterned surface, for example surface 820, optionally a surface of an electrode, comprises a plurality of recesses, for example stripes 822, shaped and sized to group and align muscle cells to form aligned myotubes. In some embodiments, at least one electrode, optionally at least two spaced-apart electrodes, for example electrodes 824, 826 and 828 cross at least some of the stripes 822, for example to record electric activity from at least some muscle cells or myotubes.

According to some exemplary embodiments, for example as shown in FIG. 8F, a micro-patterned surface, for example surface 840, optionally a surface of an electrode, comprises a plurality of recesses, for example stripes 642, shaped and sized to group and align muscle cells to form aligned myotubes. In some embodiments, at least one electrode is placed under a single recess, for example recess 849 to record the electric activity of a single myotube cultured within the recess 849. In some embodiments, a plurality of electrodes, for example electrodes 846 and 844 are positioned within some of the recesses of the plurality of recesses, for example to record the electric activity of selected myotubes cultured in the recesses.

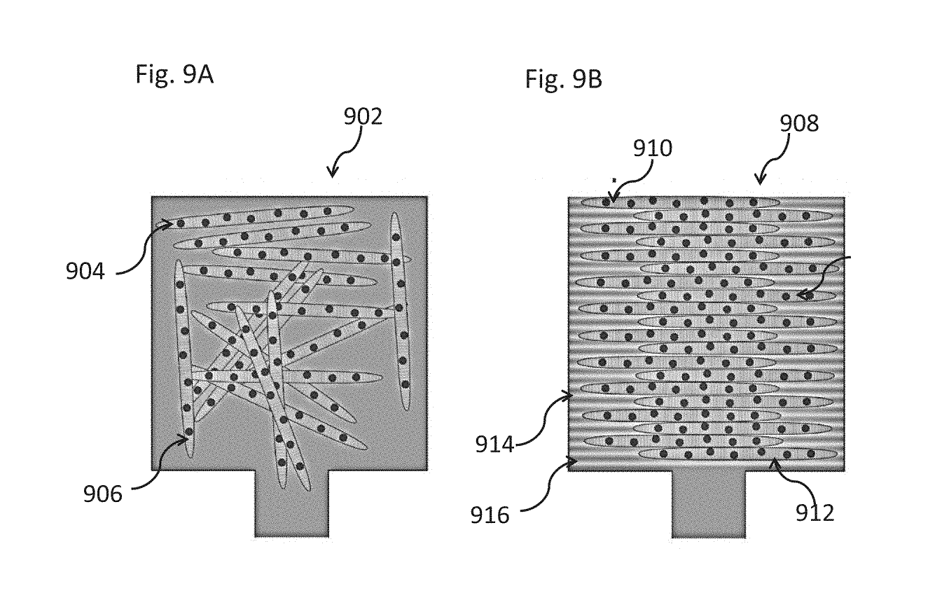

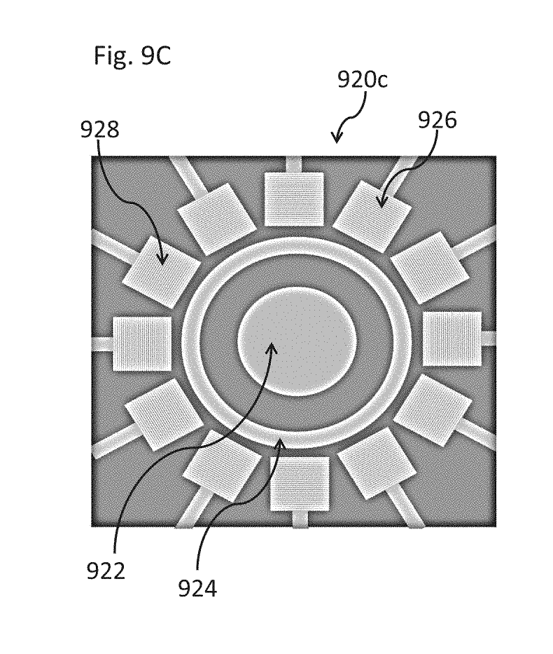

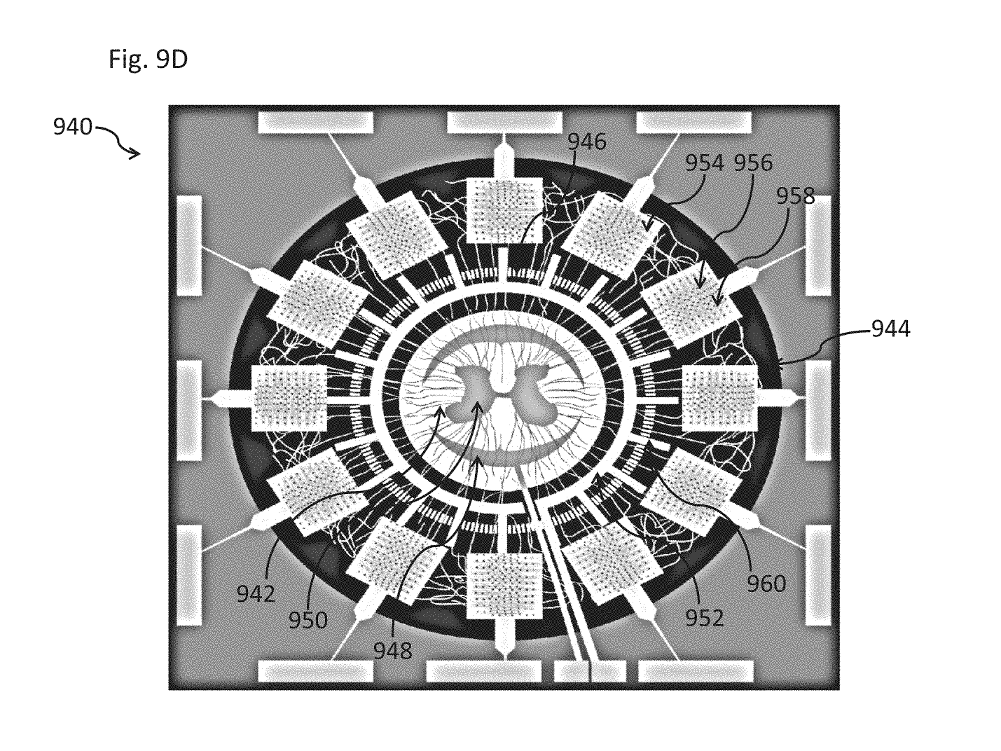

Reference is now made to FIG. 9A depicting a flat surface electrode and to FIG. 9B depicting a micro-patterned electrode, according to some exemplary embodiments of the invention.

According to some exemplary embodiments, for example as shown in FIG. 9A, muscle cells attached to the smooth surface of electrode 902 form muscle fibers, for example muscle fibers 904 and 906 in a random orientation. In some embodiments, electrical fields generated by each of the muscle fibers mask and optionally interfere and/or cancel electric fields generated by adjacent muscle fibers on the smooth surface of the electrode 902.

According to some exemplary embodiments, for example as shown in FIG. 9B, muscle cells attached to a micro-patterned surface of the micro-patterned electrode 908 for aligned muscle fibers, for example muscle fibers 910 and 912 in gaps 916 and/or in stripes 914 on the micro-patterned surface.

Exemplary Analytic Unit with Micro-Patterned Electrodes

Reference is now made to FIG. 9C depicting an electrode array of an analytic unit with a plurality of micro-patterned electrodes, according to some exemplary embodiments of the invention.

According to some exemplary embodiments, an electrode array, for example electrode array 920 comprises at least one central electrode, for example electrode 922. In some embodiments, the at least one central electrode is round, oval, arc shaped or shaped as a rectangular. In some embodiments, the central electrode is shaped and sized to be placed within a central cell culturing chamber, for example the central chamber 332 shown in FIG. 3C. In some embodiments, the central electrode is used to record the electric activity of cells, for example neuronal cells cultured in the central cell culturing chamber. Alternatively or additionally, the central electrode is used to deliver an electric field to the cells within the central chamber.

According to some exemplary embodiments, the electrode array 920 comprises at least one ring or arc-shaped electrode, for example electrode 924 shaped and sized to be positioned under at least some of the microgrooves, for example microgrooves 334 shown in FIGS. 3C-3E. Optionally, the electrode 924 is used for recording electrical activity of axons originating from the central cell culturing chamber. Alternatively or additionally, the electrode is used to deliver an electric field to at least some of the axons within the microgrooves.

According to some exemplary embodiments, the electrode array 920 comprises at least one peripheral electrode, for example electrodes 928 and 926, electrically isolated from the central electrode 922 and the ring electrode 924. Optionally at least some of the peripheral electrodes are micro-patterned electrodes, for example electrode 810 shown in FIG. 8B. In some embodiments, some of the micro-patterned electrodes comprise at least two electrically isolated electrode contacts positioned in two or more locations on the electrode surface, for example in stripes or gaps, for example as shown in FIGS. 8A-8F and in FIGS. 9A-9B for recording the electrical activity of selected one or more muscle fibers. In some embodiments when the peripheral electrode comprises one or more electrode contacts, the surface of the electrode surrounding the electrode contacts is made from a non-conductive material. In some embodiments, each of the peripheral electrodes 926 and 928 includes a separate electrical wiring. Alternatively, some of the peripheral electrodes share the same electrical wiring.

Reference is now made to FIG. 9D depicting an analytic unit which comprises spaced apart cell culturing chambers and at least one electrode array, according to some exemplary embodiments of the invention.

According to some exemplary embodiments, analytic unit 940 comprises a central cell culturing chamber 942, and a spaced-apart surrounding chamber 944, interconnected by a series of microgrooves 946. In some embodiments, at least one arc-shaped electrode 948 is placed within the central chamber. In some embodiments, at least one ring electrode, for example ring electrode 952 is positioned within the central chamber 942 between the arc shaped electrode 948 and the microgrooves 946. In some embodiments, two or more peripheral electrodes, for example electrodes 954 and 956, optionally micro-patterned electrodes, are positioned within the surrounding chamber 944.

According to some exemplary embodiments, a spinal cord explant 950 is cultured within the central chamber 942. In some embodiments, axons emanating from the explant extend through the microgrooves 946 into the surrounding chamber 944. In some embodiments, some of the axons reach one or more aligned muscle fibers 958 cultured on top the micro-patterned electrodes, and optionally form NMJ with one or more of the muscle fibers on the electrode.

According to some exemplary embodiments, the electrical activity of the muscle cells and/or muscle fibers is recorded using one or more of the electrodes 954 and/or 956. Optionally, the electrical activity of the muscle cells and/or the muscle fibers is recorded before, during and/or following application of an electric field through at least one electrode, for example arc electrode 948 to cells in the explant 950. Alternatively the electric field is delivered through the ring electrode 952 to axons emanating from the explant.

### Exemplary Electrical Recordings

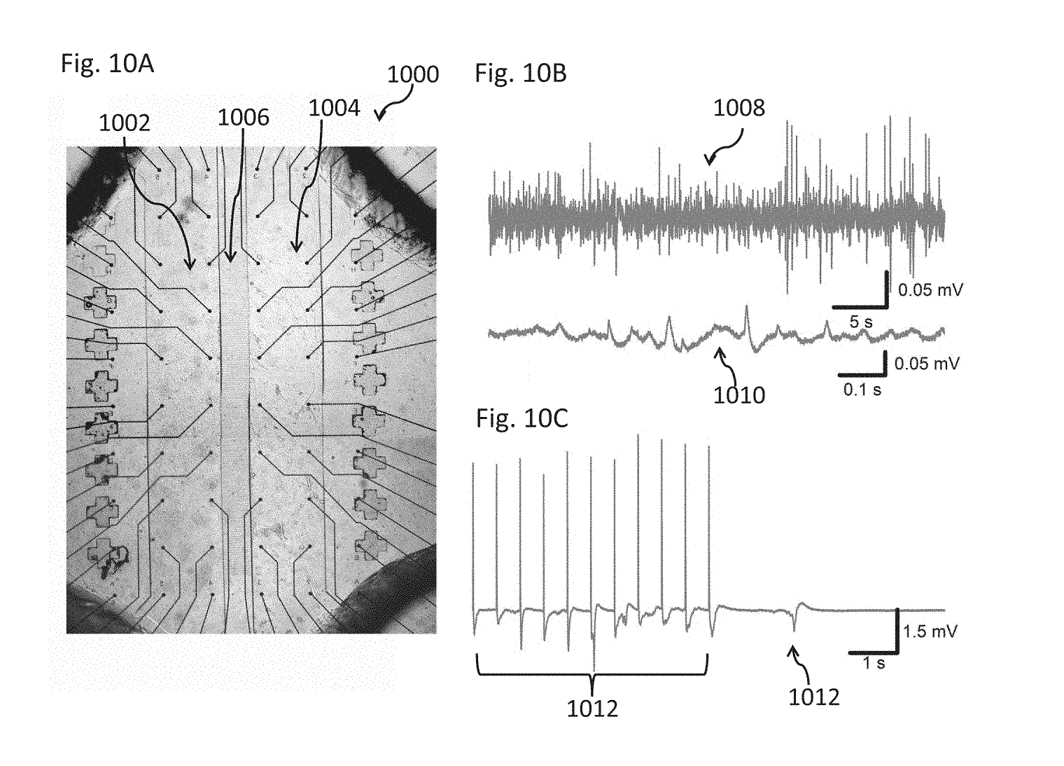

Reference is now made to FIGS. 10A-10C depicting electrical recordings from Muscle-Motor Neurons Co-culture in a Microfluidic Chamber Layered on a Multielectrode Array Chip demonstrating spontaneous and evoked activity of the myocytes, according to some exemplary embodiments of the invention.

According to some exemplary embodiments, for example as shown in the light microscopy image in FIG. 10A, a microfluidic chamber is layered on top of a 6×10 mm multielectrode array. In some embodiments, dissociated motor neuron culture plated in the right compartment of the microfluidic chamber 1000 on top of 20 electrodes. Additionally, myocyte culture is plated in the left compartment 1002 on top of 20 electrodes. In some embodiments, the muscle and neuron compartments are spaced by parallel 300 μm-long microgrooves 1006. In some embodiments, 20 electrodes in the right chamber 1004 are used for stimulation of neuronal culture by generation of electric field. In some embodiments, 20 electrodes within the left chamber 1002 are used for reading electric fields generated by muscles in the left chamber 1002.

According to some exemplary embodiments, recordings of electrical activity of the muscle cells without applying an electric field to the neurons reveal spontaneous endplate potentials, as shown for example in recording 1008 which is a 43 seconds of recordings 1008, or in recording 1010, which is a 1 sec recording section.

According to some exemplary embodiments, for example as shown in FIG. 10C, an electric field is delivered to the neurons. In some embodiments, the electric field is delivered in a single train of 10 pulses of 2 Hz. In some embodiments, the electric field is delivered with intensity in a range of 30-100 uA. In some embodiments, in response to the delivered electric filed, 10 stimulation artifacts 1012 were recorded from the myocytes followed by evoked endplate potentials 1012.

It is expected that during the life of a patent maturing from this application many relevant electrode arrays for measuring electric properties of NMJ will be developed; the scope of the term NMJ-MEA is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A microfluidic chamber, comprising:
an internal compartment sized and shaped for culturing cells;
an external compartment surrounding at least part of said internal compartment, sized and shaped for culturing cells;
at least one flow path connecting said internal compartment and said external compartment, sized and shaped to allow penetration of cell extensions from said internal compartment into said external compartment, wherein said internal compartment, said external compartment and said at least one flow path are formed on a base layer of said microfluidic chamber;
at least one micro-pattern in said base layer of said microfluidic chamber shaped and sized to align cells cultured in said microfluidic chamber relative to each other.

2. The microfluidic chamber of claim 1, wherein said at least one micro-pattern is positioned in said base layer of said at least one flow path and comprising a plurality of microgrooves shaped and sized to align said cell extensions penetrating through said microgrooves from said internal compartment to said external compartment.

3. The microfluidic chamber of claim 2, wherein said external compartment comprises a micro-patterned surface configured to align cells cultured in said external compartment relative to each other and/or relative to said cell extensions.

4. The microfluidic chamber of claim 3, wherein said micro-patterned surface of said external compartment comprises a plurality of elongated recesses shaped and sized to group muscle cells to form aligned myotubes, wherein at least some of said elongated recesses are parallel relative to each other.

5. The microfluidic chamber of claim 4, wherein a width of said recesses is in a range of 2-10 micron, and wherein a length of said recesses is in a range of 50-2000 micron.

6. The microfluidic chamber of claim 4, wherein said plurality of microgrooves are shaped and sized to allow passage of axons emanating from neuronal cells cultured in said internal compartment towards said myotubes cultured in said external compartment.

7. The microfluidic chamber of claim 3, comprising at least one electrode associated with said external compartment, wherein said electrode comprises said micro-patterned surface and is sized and positioned to measure electric properties of a cell population cultured in said external compartment.

8. The microfluidic chamber of claim 1, wherein said external compartment is shaped as an arc surrounding at least 180 degrees of said internal compartment.

9. The microfluidic chamber of claim 8, wherein said electrode is an arc-shaped electrode.

10. The microfluidic chamber of claim 1, further comprising at least one electrode associated with said internal compartment for applying an electric field to cells cultured in said internal compartment.

11. The microfluidic chamber of claim 2, further comprising an additional set of electrodes associated with said microgrooves for measuring electric properties of said cell extensions cultured in said microgrooves, wherein said set of electrodes is electrically isolated from other electrodes of said microfluidic chamber.

12. The microfluidic chamber of claim 11, wherein said set of electrodes is positioned between said electrode of said internal compartment and said electrode of said external compartment.

13. The microfluidic chamber of claim 2, wherein said internal compartment is sized and shaped for culturing spinal cord explants or motor neurons.

14. The microfluidic chamber of claim 13, wherein a cross-section of said microgrooves is sized for selective penetration of neuronal extensions emanating from said motor neurons into said microgrooves.

15. The microfluidic chamber of claim 1, wherein said external compartment is sized and shaped for culturing muscle cells, myotubes, glia cells, glandular cells or neurons.

16. The microfluidic chamber of claim 2, wherein a length of said microgrooves is in a range of 50-700 μm and a width of said microgrooves is in a range of 1-10 μm.

17. The microfluidic chamber of claim 1, wherein a bottom surface of said internal compartment and/or said external compartment is coated with at least one organic material to increase cell adhesion to said bottom surface and wherein said organic material is selected from a list of laminin, fibronectin, poly-1-lysine, poly-1-ornithine or matrigel.

18. The microfluidic chamber of claim 1, wherein said microfluidic chamber is round and/or shaped and sized to be positioned within a cell culturing plate.

19. The microfluidic chamber of claim 1, wherein said microfluidic chamber is shaped and sized to be positioned within a well of at least 2-well cell culturing plate.

20. The microfluidic chamber of claim 1, comprising living cells and/or cell culturing media.

21. A microfluidic chamber, comprising:
at least two spaced-apart compartments sized and shaped for culturing cell populations;
a plurality of microgrooves in the base layer of said chamber connecting a first compartment of said compartments with a second compartment of said compartments, wherein said microgrooves are sized and shaped to allow cell extensions from said first compartment to penetrate into said second compartment;
at least three electrodes configured and positioned to measure electric properties and/or to apply an electric field, wherein a first electrode of said three electrodes is associated with said microgrooves for measuring electric properties of a plurality of said cell extensions and wherein a second electrode is associated with said first compartment and a third electrode is associated with said second compartment.

22. A method for measuring electric properties of cultured muscle cells, comprising:
culturing neuronal cells and muscle cells in two spaced-apart compartments; and
measuring electric properties of said muscle cells by a first electrode, and electric properties of neuronal extensions extending from said neurons towards said muscle cells or electric properties of said neuronal cells by at least one second electrode.

23. The method of claim 22, comprising:
applying an electric field to said neuronal cells before said measuring.

24. The method of claim 23, wherein said measuring comprises measuring the electric properties of said muscle cells and/or the neuronal extensions in response to said electric field.

25. The method of claim 22, comprising:
providing at least one bioactive agent to muscle cells before said measuring;
determining the effect of said at least one bioactive agent on said muscle cells based on said measuring.

26. The method of claim 22, wherein said culturing comprises culturing said muscle cells within elongated recesses to form aligned and parallel myotubes relative to each other, and wherein measuring comprises measuring electric properties of at least some of said aligned and parallel myotubes.

27. A method for screening materials capable of restoring synaptic function, comprising:
providing a first cell population and a second separated cell population, wherein said first cell population is capable of forming synapses with said second separated cell population;
treating said first cell population and/or said second separated cell population with at least one material of said materials;
measuring electric properties of said first cell population and/or of said second separated cell population;
determining functionality of said synapses between said first cell population and said second separated cell population based on the results of said measuring; and
identifying said material for restoring functionality of said synapses based on said determining.

28. The method of claim 27, wherein said treating comprises treating said first cell population and/or said second separated cell population at least twice, each with a different dosage of said material, and wherein identifying comprising identifying said dosage of said material capable of restoring functionality of said synapses.

29. The method of claim 27, wherein said treating comprises treating said first cell population and/or said second separated cell population at least twice, each with a different treatment regime of said material, and wherein identifying comprising identifying said treatment regime of said material capable of restoring functionality of said synapses.

30. The method of claim 27, wherein said first cell population and a second separated cell population are cultured for a desired time period for forming said synapses prior to said measuring.
